(19) United States
(12) Patent Application Publication
(10) Pub. No.: US 2014/0155470 A1
(43) Pub. Date: Jun. 5, 2014

(54) PLA2R1 AS ANTI-TUMORAL COMPOUND AND AS BIOMARKER FOR THE DETECTION OF CANCER

(71) Applicants: David Bernard, Trept (FR); Arnaud Augert, Seattle (FR); Gerard Lambeau, Grasse (FR); Christophe Girard, Nice (FR); David Vindrieux, Saint-Etienne (FR)

(72) Inventors: David Bernard, Trept (FR); Arnaud Augert, Seattle (FR); Gerard Lambeau, Grasse (FR); Christophe Girard, Nice (FR); David Vindrieux, Saint-Etienne (FR)

(21) Appl. No.: 14/131,267

(22) PCT Filed: Jul. 6, 2012
(86) PCT No.: PCT/EP2012/063272

§ 371 Date: Jan. 7, 2014

Related U.S. Application Data
(60) Provisional application No. 61/505,887, filed on Jul. 8, 2011.

(30) Foreign Application Priority Data
Jul. 8, 2011 (EP) 11305885.3

(51) Int. Cl. A61K 38/17; C07K 14/705

(52) U.S. Cl. CPC A61K 38/178; C07K 14/7056
USPC 514/44 R; 530/396; 536/23.5

(57) ABSTRACT

Compositions comprising a polynucleotide of human PLA2R1, a polypeptide of human PLA2R1 or an extracellular fragment of human PLA2R1 for use in the treatment of cancer in particular breast cancer, pancreatic cancer, colorectal cancer, kidney cancer and melanoma.

Cancer incidence increases because of the aging population and of changes in the life style in developing countries. More than 10 million cancers are detected worldwide each year and about 7 million people died of cancer. Cancers are now representing about 13% of all deaths each year. Breast cancer is the most frequent cancer for women in western countries and still represents about 13% of cancer death for women (and 6% with male included). Pancreatic and kidney cancers are less frequent. But their incidence in term of mortality is high. Indeed, except surgery that can be performed only in the early stages of the disease (most cancers being detected at late stages), the other treatments are frequently inefficient and most of the time do not increase significantly the patient survival. Therefore, there is still a need for new and efficient anti-cancer treatments in particular for treating adenocarcinomas such as breast cancer, pancreatic cancer and kidney cancer.

Cancer cells derive from normal cells that accumulate genetic and epigenetic alterations. During this process and among other steps, normal cells acquire immortality and enhanced stress resistance (Hanahan and Weinberg, 2000). Restoring genetic events lost during carcinogenesis process may thus exert anti-tumoral activity.

PLA2R1 is a large glycosylated membrane protein of 180 kDa which is also known as the M-type sPLA2 receptor. It belongs to the superfamily of C-type lectins, and its closest paralogs are the macrophage mannose receptor, the endo-180/UPARAP protein and the DEC-205 (Engelholm et al., 2009; Shrimpton et al., 2009; Taylor et al., 2005). PLA2R1 is a type I receptor comprising a single transmembrane domain, a short cytoplasmic tail, and a very large extracellular region made up of an N-terminal cysteine-rich domain, a fibronectin-like type II domain, and a tandem repeat of eight distinct C-type lectin-like carbohydrate recognition domains (CTLDs). It also exists as a soluble secreted receptor comprising the full extracellular region. PLA2R1 is expressed in several tissues including lung, kidney, spleen and colon. It was originally discovered two decades ago in skeletal muscle cells (M-type receptor) by virtue of binding of various venom and mammalian secreted phospholipases A2 (sPLA2s) venom. Since then, PLA2R1 has been proposed to mediate several in vitro functions of sPLA2s and to play a pro-inflammatory role in a mouse model of septic shock. However, PLA2R1 is likely a multifunctional receptor for which the multiple set of in vivo biological functions and the diversity of endogenous ligands still remain largely unknown (Lambeau and Lazdunski, 1999; Murakami et al., 2010).

Cellular senescence results in an irreversible proliferation arrest. Concomitantly, specific markers appear in senescent cells. This cellular response may be involved in various physiopathological conditions especially during aging and aging-related diseases (Campisi, 2011; Rodier and Campisi, 2011). Cellular senescence, by inducing a proliferation arrest is proposed to exert anti-tumoral effect at early stage of tumoral development and during anti-tumoral treatment (Braig et al., 2005; Chen et al., 2005; Collado et al., 2005; Ewald et al., 2010).

We have previously shown that a down-regulation of PLA2R1 favors cellular senescence escape whereas its ectopic expression in normal cells induces cellular senescence (Augert et al., 2009). However, any effect of PLA2R1 on cancer cell lines was neither described nor suggested.

WO2005/104810 describes putative cancer-associated sequences or genes which have been identified by the use of oncogenic retroviruses whose sequences insert into the genome of a host organism resulting in cancer (see paragraphs [0041]-[0042] of WO2005/104810). This document contains no experimental data or teaching demonstrating that insertion in PLA2R1 sequence is linked to cancer occurrence, demonstrating any therapeutic effect of these sequences or of any putative polypeptide encoded by these sequences.

It is now surprisingly shown that PLA2R1 also induces cell death in various cancer cell lines. Further, PLA2R1 levels decrease in cancer cell lines and PLA2R1 is therefore a biomarker for the detection of cancer.

### SUMMARY

The present invention is related to compositions comprising a polynucleotide selected in the group consisting of:
The polynucleotide of SEQ ID NO. 1 or a variant thereof having at least 95% identity with the sequence of SEQ ID No. 1;
A polynucleotide encoding the human PLA2R1 polypeptide of SEQ ID NO. 2 or encoding a variant thereof having at least 95% identity with the sequence of SEQ ID No. 2,
A polynucleotide from position 1 to position 4191 of SEQ ID NO.1 encoding the extracellular domain of human PLA2R1 or a variant thereof having at least 95% identity with the polynucleotide from position 1 to position 4191 of SEQ ID NO.1;
A fragment of the polynucleotide from position 1 to position 4191 of SEQ ID NO.1 encoding a fragment of the extracellular domain of PLA2R1 or a variant thereof having at least 95% identity with a fragment of the polynucleotide from position 1 to position 4191 of SEQ ID NO.1;
A polynucleotide encoding a polypeptide of SEQ ID NO. 4, 6 or 8; for use in a method for the treatment of cancer.

Preferably, the fragment of the polynucleotide from position 1 to position 4191 of SEQ ID NO.1 encodes at least the Cys-rich domain, the fibronectin type II domain and a C-type lectin domain of human PLA2R1.

More preferably, the fragment of the polynucleotide from position 1 to position 4191 of SEQ ID NO.1 is selected in the group consisting of the polynucleotides of SEQ ID NO. 3, SEQ ID NO. 5, SEQ ID NO. 7 and variants thereof having at least 95% identity with the polynucleotide of SEQ ID NO. 3, SEQ ID NO.5 or SEQ ID NO.7.

In a first embodiment the polynucleotide is inserted in a viral vector. In another embodiment, the polynucleotide is incorporated in a delivery vehicle.

Another object of the present invention is a composition comprising a polypeptide selected in the group consisting of:
The Human PLA2R1 polypeptide depicted in SEQ ID NO. 2 or a variant thereof having at least 95% identity with the polypeptide of SEQ ID No. 2;
The extracellular domain of human PLA2R1 polypeptide from position 21 to position 1397 of SEQ ID NO.2 or a variant thereof having at least 95% identity with the human PLA2R1 polypeptide from position 21 to position 1397 of SEQ ID NO.2;
A fragment of the extracellular domain of human PLA2R1 polypeptide from position 21 to position 1397 of SEQ ID NO.2 or a variant thereof having at least 95% identity with a fragment the human PLA2R1 polypeptide from position 21 to position 1397 of SEQ ID NO.2;

for use in a method for the treatment of cancer.

Preferably, the fragment of the extracellular domain of human PLA2R1 polypeptide from position 21 to position 1397 of SEQ ID NO.2 comprises at least the Cys-rich domain, the fibronectin type II domain and a C-type lectin domain of human PLA2R1.

More preferably, the fragment of the extracellular domain of human PLA2R1 polypeptide from position 21 to position 1397 of SEQ ID NO.2 is selected in the group consisting of the polypeptide from position 21 to position 1322 of SEQ ID NO. 4, the polypeptide from position 21 to position 1098 of SEQ ID NO. 6, the polypeptide from position 21 to position 645 of SEQ ID NO. 7 and variants thereof having at least 95% identity with the polypeptide from position 21 to position 1322 of SEQ ID NO. 4, at least 95% identity with the polypeptide from position 21 to position 1098 of SEQ ID NO. 6 or at least 95% identity with the polypeptide from position 21 to position 645 of SEQ ID NO. 8.

In preferred embodiments, the compositions of the present invention are for use in a method for the treatment of breast cancer, pancreatic cancer, kidney cancer, colorectal cancer or melanoma.

Another object of the present invention is a polypeptide encoding a soluble extracellular fragment of human PLA2R1 consisting of the polypeptide of SEQ ID NOs. 4, 6, 8 or variants thereof having at least 95% identity with the polypeptide of SEQ ID NO. 4, at least 95% identity with the polypeptide of SEQ ID NO. 6 or at least 95% identity with the polypeptide of SEQ ID NO. 8.

Another object of the present invention is a polypeptide according to the present invention for use in a method for the treatment of cancer more particularly for use in a method for the treatment of breast cancer, kidney cancer, pancreatic cancer, colorectal cancer and/or melanoma.

Another object of the present invention is a polynucleotide encoding a polypeptide according to the present invention. Preferably, the polynucleotides according to the present invention are for use in a method for the treatment of cancer, more particularly for use in the treatment of breast cancer, kidney cancer, pancreatic cancer, colorectal cancer and/or melanoma.

The present invention is also related to a method for detecting a cancer in a patient comprising the following steps:
Measuring the level of expression of the PLA2R1 gene in a sample previously taken from a patient,
Comparing the expression level of the PLA2R1 gene with the expression level of the PLA2R1 gene in a control sample,
   wherein a decrease of the PLA2R1 expression level in the sample previously taken from the patient compared to the level of expression of the PLA2R1 gene in the control sample is significant of the presence of a cancer.

Preferably, the method is for detecting breast cancer, kidney cancer, pancreatic cancer, colorectal cancer or melanoma. More preferably, the method is for detecting breast cancer or a kidney cancer.

Another object of the present invention is a method for determining the prognosis of a cancer in a patient comprising the following steps:
a) Measuring the level of expression of the PLA2R1 gene in cancer cells from a tumour sample previously taken from said patient,
b) Classifying the cancer as having a poor prognosis if the PLA2R1 gene is under-expressed in said cancer cells.

Preferably, the method is for detecting breast cancer, kidney cancer, pancreatic cancer, colorectal cancer or melanoma. More preferably, the method is for detecting breast cancer or a kidney cancer.

### SEQUENCE LISTING

SEQ ID No.1: PLA2R1 nucleotide sequence
SEQ ID No.2: PLA2R1 amino acid sequence
SEQ ID No.3: PLA2R1 CysR-CTLD8 nucleotide sequence
SEQ ID No.4: PLA2R1 CysR-CTLD8 amino acid sequence
SEQ ID No.5: PLA2R1 CysR-CTLD6 nucleotide sequence
SEQ ID No.6: PLA2R1 CysR-CTLD6 amino acid sequence
SEQ ID No.7: PLA2R1 CysR-CTLD3 nucleotide sequence
SEQ ID No.8: PLA2R1 CysR-CTLD3 amino acid sequence
SEQ ID No.9: PLA2R1 CysR-FNIIs nucleotide sequence
SEQ ID No.10: PLA2R1 CysR-FNIIs amino acid sequence
SEQ ID No.11: Forward primer
SEQ ID No.12: WT reverse primer
SEQ ID No.13: FNII reverse primer
SEQ ID No.14: CTLD3 reverse primer
SEQ ID No.15: CTLD6 reverse primer
SEQ ID No. 16: CTLD8 reverse primer

### DETAILED DESCRIPTION OF THE INVENTION

The present invention relates to polynucleotides and polypeptides derived from human PLA2R1 (Phospholipase A2 receptor 1) and their use in methods for the treatment of cancer in particular for the treatment of breast cancer, kidney cancer, pancreatic cancer, colorectal cancer and melanoma. It has been surprisingly found that expression of polynucleotides encoding PLA2R1 in tumor cells triggers cell death in these tumor cells.

The term “polynucleotide” according to the present invention refers to a single strand nucleotide chain or its complementary strand which can be of the DNA or RNA type, or a double strand nucleotide chain which can be of the cDNA (complementary) or genomic DNA type. The term “polynucleotide” also refers to modified polynucleotides.

The polynucleotides of this invention are isolated or purified from their natural environment. Preferably, the polynucleotides of this invention can be prepared using conventional molecular biology techniques such as those described by Sambrook et al. (Molecular Cloning: A Laboratory Manual, 1989) or by chemical synthesis.

In a first embodiment, the present invention relates to the polynucleotide of SEQ ID NO. 1 or variants thereof having at least 95% identity with the sequence of SEQ ID No.1 for use in the treatment of cancer. The polynucleotide of SEQ ID NO. 1 encodes the human PLA2R1 (phospholipase A2 receptor 1) polypeptide.

The polynucleotide of SEQ ID NO. 1 encodes the different domains of the PLA2R1 polypeptide, the positions on the sequence of SEQ ID NO. 1 are as follows:

nucleotides 1-60 signal peptide,

nucleotides 112-483 Cysteine rich domain (CysR),

nucleotides 517-663 Fibronectin type II domain (FNII),

nucleotides 712-1065 C-type lectin 1 domain (CTLD1),

nucleotides 1153-1506 C-type lectin 2 domain (CTLD2),

nucleotides 1564-1929 C-type lectin 3 domain (CTLD3),

nucleotides 2017-2391 C-type lectin 4 domain (CTLD4),

nucleotides 2455-2814 C-type lectin 5 domain (CTLD5),

nucleotides 2893-3288 C-type lectin 6 domain (CTLD6),

nucleotides 3361-3696 C-type lectin 7 domain (CTLD7),

nucleotides 3769-4134 C-type lectin 8 domain (CTLD8),

nucleotides 4192-4254 transmembrane domain,

nucleotides 4255-4389 intracellular domain.

Further, the nucleotides from position 61 to 4191 encode the extracellular domain of the mature protein after clivage of the signal peptide.

The invention is also related to a polynucleotide encoding the human PLA2R1 polypeptide of SEQ ID NO. 2 or a variant thereof having at least 95% identity with the sequence of SEQ ID No. 2 for use in a method for the treatment of cancer.

It has also been found that the expression of the soluble extracellular domain of PLA2R1 or of a fragment thereof in tumour cells triggers cell death in these cells.

In a preferred embodiment, the present invention relates to a polynucleotide from position 1 to position 4191 of SEQ ID NO.1 encoding the extracellular domain of human PLA2R1 or a variant thereof having at least 95% identity with the polynucleotide from position 1 to position 4191 of SEQ ID NO.1.

In another preferred embodiment, the present invention is related to a fragment of the polynucleotide from position 1 to position 4191 of SEQ ID NO.1 encoding a fragment of the extracellular domain of PLA2R1 or a variant thereof having at least 95% identity with a fragment of the polynucleotide from position 1 to position 4191 of SEQ ID NO.1.

Preferably the fragment of the polynucleotide from position 1 to position 4191 of SEQ ID NO.1 encodes at least the Cys-rich domain, the fibronectin type II domain and a C-type lectin domain of human PLA2R1. The positions of these domains on the sequence of SEQ ID NO. 1 are indicated above.

In preferred embodiments, the fragment of the polynucleotide from position 1 to position 4191 of SEQ ID NO.1 is selected in the group consisting of the polynucleotides of SEQ ID NO. 3, SEQ ID NO. 5, SEQ ID NO. 7 and variants thereof having at least 95% identity with the polynucleotide of SEQ ID NO. 3, at least 95% identity with the polynucleotide of SEQ ID NO.5 or at least 95% identity with the polynucleotide of SEQ ID NO.7.

The polynucleotide of SEQ ID NO. 3 corresponds to positions 1-3966 of SEQ ID NO. 1. This polynucleotide is a fragment of the extracellular domain of PLA2R1 and it comprises the sequences encoding the signal peptide, the Cys R domain, the FNII domain and the CTLD domains 1-8 as described above.

The polynucleotide of SEQ ID No. 5 corresponds to positions 1-3294 of SEQ ID NO. 1. This polynucleotide is a fragment of the extracellular domain of PLA2R1 and it comprises the sequences encoding the signal peptide, the Cys R domain, the FNII domain and the CTLD domains 1-6 as described above.

The polynucleotide of SEQ ID No. 7 corresponds to positions 1-1935 of SEQ ID NO. 1. This polynucleotide is a fragment of the extracellular domain of PLA2R1 and it comprises the sequences encoding the signal peptide, the Cys R domain, the FNII domain and the CTLD domains 1-3 as described above.

The present invention is also directed to polynucleotides encoding the extracellular domain of human PLA2R1 of SEQ ID NO. 1 or fragments thereof, in particular to polynucleotides encoding the polypeptides of SEQ ID NOs. 4, 6 or 8.

The invention also relates to variants of the polynucleotides described above wherein said variants present a degree of identity with the polynucleotides described above. Preferably, these variants are naturally occurring variants found in the human population.

The invention thus relates to variants of the polynucleotides described above presenting at least 90%, 95%, 98% and preferably at least 99% identity with these polynucleotides. The term identical polynucleotides refers to nucleotides with no variation or changes between two sequences. These polynucleotides can have a deletion, addition or substitution of at least one nucleotide with respect to the reference polynucleotide. Preferably, the variants of the polynucleotides described above present at least 90%, 95%, 98% and preferably at least 99% identity with these polynucleotides over their whole length.

Preferably, the variants of the polynucleotides described above retain the properties of the polynucleotide from which they are derived. In particular, the polynucleotides of the present invention trigger cell death in tumor cells when they are expressed in these cells.

The term polynucleotide “fragments” refers to a polynucleotide including part but not all of the polynucleotide from which it is derived. The fragments according to this invention retain the properties of the polynucleotide from which they are derived. In particular, the polynucleotides of the present invention trigger cell death in tumor cells when they are expressed in these cells.

The degree of identity between two sequences, quantified by a score, is based on the percentage of identities and/or changes in the sequences. The methods for measuring and identifying the degree of identity between nucleic acid sequences are well known to the man skilled in the art. For example, vectors NTi Vector NTi 9.1.0, alignment program AlignX (Clustal W algorithm) (Invitrogen INFORMAX) can be used. Preferably, the default parameters are used.

The invention also relates to polynucleotides capable of selective hybridization with the polynucleotides described above. Preferably, these polynucleotides retain the properties of the polynucleotide to which they hybridize. In particular, the polynucleotides of the present invention trigger cell death in tumor cells when they are expressed in these cells.

Preferably, selective hybridization is carried out under average conditions of stringency and even more preferably under strict conditions of stringency. The term “sequence capable of selective hybridization” according to the invention refers to sequences which hybridize with the reference sequence at a significantly higher level than background noise. The level of the signal generated by interaction between the sequence capable of selective hybridization and the reference sequence is generally 10 times, preferably 100 times more intense than that for the interaction with other DNA sequences which generate background noise. The strict hybridization conditions leading to selective hybridization are well known to the man skilled in the art. In general, the hybridization and washing temperature is 5° C. below the Tm of the reference sequence at a given pH and for a given ionic force. Typically, the hybridization temperature is at least 30° C. for a 15 to 50 nucleotide polynucleotide and at least 60° C. for a polynucleotide with over 50 nucleotides. As an example, hybridization is carried out in the following buffer: 6×SSC, 50 mM Tris-HCI (pH 7.5), 1 mM EDTA, 0.02% PVP, 0.02% Ficoll, 0.02% BSA, 500 ug/ml of denatured salmon sperm DNA. Washing is carried out, for example, successively at low stringency in 2×SSC, 0.1% SDS buffer, at average stringency in 0.5×SSC, 0.1% SDS buffer and at high stringency in 0.1×SSC, 0.1% SDS. Hybridization can evidently be carried out using other methods well known to the man skilled in the art (see in particular Sambrook et al., Molecular Cloning: A Laboratory Manual, 1989). Preferably, the polynucleotides which hybridize selectively with a reference polynucleotide retain the function of the reference sequence.

In preferred embodiments, the polynucleotides described above are inserted in a viral vector or a pseudoviral vector. Preferably, the vector is suitable to protect and deliver the polynucleotides described above across the cell membrane into the nucleus, to enable their expression in a specific target cell and more particularly in a tumour cell. This viral vector is suitable for targeting and expression in tumor cells for use in the treatment of cancer. Vectors for use in the methods of the present invention are for example retroviruses or adenoviruses.

In other embodiments, the polynucleotides described above are incorporated in a delivery vehicle and more particularly in a non-viral delivery vehicle. To improve the targeting and expression of a polynucleotide in a tumor cell, the polynucleotide must be protected and delivered to the nucleus of the target cell. Any suitable delivery vehicle may be used in the methods of the present invention including liposomes and polymers.

The present invention also relates to polypeptides derived from human PLA2R1 (Phospholipase A2 receptor 1) and their use in a method for the treatment of cancer in particular in a method for the treatment of breast cancer, kidney cancer and pancreatic cancer. The present invention surprisingly shows that the PLA2R1 polypeptide triggers cell death in tumour or cancer cells.

In a first embodiment, the present invention relates to the human PLA2R1 polypeptide depicted in SEQ ID NO. 2 or a variant thereof having at least 95% identity with the polypeptide of SEQ ID No. 2 for use in the treatment of cancer.

The polypeptide of SEQ ID NO. 2 comprises the different domains of the PLA2R1 polypeptide, the positions on the sequence of SEQ ID NO. 2 are as follows:

amino acids 1-20 signal peptide,

amino acids 38-161 Cysteine rich domain (CysR),

amino acids 173-221 Fibronectin type II domain (FNII),

amino acids 238-355 C-type lectin 1 domain (CTLD1),

amino acids 385-502 C-type lectin 2 domain (CTLD2),

amino acids 522-643 C-type lectin 3 domain (CTLD3),

amino acids 673-797 C-type lectin 4 domain (CTLD4),

amino acids 819-938 C-type lectin 5 domain (CTLD5),

amino acids 965-1096 C-type lectin 6 domain (CTLD6),

amino acids 1121-1232 C-type lectin 7 domain (CTLD7),

amino acids 1257-1378 C-type lectin 8 domain (CTLD8),

amino acids 1398-1418 transmembrane domain,

amino acids 1419-1463 intracellular domain.

Further, the amino acids from position 21 to 1397 correspond to the extracellular domain of the mature protein after clivage of the signal peptide.

The present invention also shows that the extracellular domain of the mature PLA2R1 polypeptide or fragments of the extracellular domain of the mature PLA2R1 polypeptide trigger cell death in cancer cells.

Accordingly, the present invention is also directed to the extracellular domain of human PLA2R1 polypeptide from position 21 to position 1397 of SEQ ID NO.2 or a variant thereof having at least 95% identity with the human PLA2R1 polypeptide from position 21 to position 1397 of SEQ ID NO.2.

In other embodiments, the invention relates to a fragment of the extracellular domain of human PLA2R1 polypeptide from position 21 to position 1397 of SEQ ID NO.2 or a variant thereof having at least 95% identity with a fragment the human PLA2R1 polypeptide from position 21 to position 1397 of SEQ ID NO.2.

Preferably, the fragment of the extracellular domain of human PLA2R1 polypeptide from position 21 to position 1397 of SEQ ID NO.2 comprises at least the Cys-rich domain, the fibronectin type II domain and a C-type lectin domain of human PLA2R1. The positions of these domains on the sequence of SEQ ID NO. 2 are indicated above.

In preferred embodiments, the fragment of the extracellular domain of human PLA2R1 polypeptide from position 21 to position 1397 of SEQ ID NO.2 is selected in the group consisting of the polypeptide from position 21 to position 1322 of SEQ ID NO. 4, the polypeptide from position 21 to position 1098 of SEQ ID NO. 6, the polypeptide from position 21 to position 645 of SEQ ID NO. 8 and variants thereof having at least 95% identity with the polypeptide from position 21 to position 1322 of SEQ ID NO. 4, at least 95% identity with the polypeptide from position 21 to position 1098 of SEQ ID NO. 6 or at least 95% identity with the polypeptide from position 21 to position 645 of SEQ ID NO. 8.

The polypeptide of SEQ ID NO. 4 corresponds to positions 1 to 1322 of SEQ ID NO. 2. This polypeptide is a fragment of the extracellular domain of PLA2R1 and it comprises the signal peptide, the Cys R domain, the FNII domain and the CTLD domains 1-8 as described above.

The polypeptide of SEQ ID NO. 6 corresponds to positions 1 to 1098 of SEQ ID NO. 2. This polypeptide is a fragment of the extracellular domain of PLA2R1 and it comprises the signal peptide, the Cys R domain, the FNII domain and the CTLD domains 1-6 as described above.

The polypeptide of SEQ ID NO. 8 corresponds to positions 1 to 645 of SEQ ID NO. 2. This polypeptide is a fragment of the extracellular domain of PLA2R1 and it comprises the signal peptide, the Cys R domain, the FNII domain and the CTLD domains 1-3 as described above.

The invention also relates to variants of the polypeptides described above wherein said variants present a degree of identity with the polypeptides described above. Preferably, these variants are naturally occurring variants found in the human population.

The invention thus relates to variants of the polypeptides described above presenting at least 90%, 95%, 98% and preferably at least 99% identity with these polypeptides. These polypeptides can have a deletion, addition or substitution of at least one amino acid with respect to the reference polypeptide. Preferably, the variants of the polypeptides described above present at least 90%, 95%, 98% and preferably at least 99% identity with these polypeptides over their whole length.

The term identical polypeptides refers to polypeptides with no variation or changes between two sequences.

Preferably, the variants of the polypeptides described above retain the properties of the polypeptides from which they are derived. In particular, the polypeptides of the present invention trigger cell death in tumor cells.

The term polypeptide “fragments” refers to a polypeptide including part but not all of the polypetide from which it is derived. The fragments according to this invention retain the properties of the polypeptide from which they are derived. In particular, the polypeptides of the present invention trigger cell death in tumor cells when they are expressed in these cells.

The degree of identity between two sequences, quantified by a score, is based on the percentage of identities and/or changes in the sequences. The methods for measuring and identifying the degree of identity between amino acid sequences are well known to the man skilled in the art. For example, vectors NTi Vector NTi 9.1.0, alignment program AlignX (Clustal W algorithm) (Invitrogen INFORMAX) can be used. Preferably, the default parameters are used.

Preferably, the polynucleotides and polypeptides described above are for use in methods for the treatment of cancer most preferably for use in methods for the treatment of breast cancer, kidney cancer and pancreatic cancer.

Another object of the present invention is a composition for use as a medicament comprising a polynucleotide or a polypeptide as described above.

The present invention is also related to compositions containing a polynucleotide or a polypeptide as described above for use in a method for the treatment of cancer in particular in the treatment of breast cancer, kidney cancer and pancreatic cancer.

The present invention encompasses pharmaceutical compositions for use in a method for the treatment of cancer in particular for the treatment of breast cancer, kidney cancer, pancreatic cancer, colorectal cancer and melanoma.

The present invention provides pharmaceutical compositions comprising:

a) an effective amount of a polynucleotide or a polypeptide as described herein, and
b) a pharmaceutically acceptable carrier, which may be inert or physiologically active.

As used herein, “pharmaceutically-acceptable carriers” includes any and all solvents, dispersion media, coatings, antibacterial and antifungal agents, and the like that are physiologically compatible. Examples of suitable carriers, diluents and/or excipients include one or more of water, saline, phosphate buffered saline, dextrose, glycerol, ethanol, and the like, as well as combination thereof. In many cases, it will be preferable to include isotonic agents, such as sugars, polyalcohols, or sodium chloride in the composition. In particular, relevant examples of suitable carrier include: (1) Dulbecco's phosphate buffered saline, pH ˜7.4, containing or not containing about 1 mg/ml to 25 mg/ml human serum albumin, (2) 0.9% saline (0.9% w/v sodium chloride (NaCl)), and (3) 5% (w/v) dextrose; and may also contain an antioxidant such as tryptamine and a stabilizing agent such as Tween 20.

The pharmaceutical compositions encompassed by the present invention may also contain a further therapeutic agent for the treatment of cancers.

The compositions of the invention may be in a variety of forms. These include for example liquid, semi-solid, and solid dosage forms, but the preferred form depends on the intended mode of administration and therapeutic application. Typical preferred compositions are in the form of injectable or infusible solutions. The preferred mode of administration is parenteral (e.g. intravenous, intramuscular, intraperinoneal, subcutaneous). In a preferred embodiment, the compositions of the invention are administered intravenously as a bolus or by continuous infusion over a period of time. In another preferred embodiment, they are injected by intramuscular, subcutaneous, intra-articular, intrasynovial, intratumoral, peritumoral, intralesional, or perilesional routes, to exert local as well as systemic therapeutic effects.

Sterile compositions for parenteral administration can be prepared by incorporating the polypeptide or the polynucleotide as described in the present invention in the required amount in the appropriate solvent, followed by sterilization by microfiltration. As solvent or vehicle, there may be used water, saline, phosphate buffered saline, dextrose, glycerol, ethanol, and the like, as well as combination thereof. In many cases, it will be preferable to include isotonic agents, such as sugars, polyalcohols, or sodium chloride in the composition.

The polypeptides or polynucleotides as described herein may also be orally administered. As solid compositions for oral administration, tablets, pills, powders (gelatine capsules, sachets) or granules may be used. In these compositions, the active ingredient according to the invention is mixed with one or more inert diluents, such as starch, cellulose, sucrose, lactose or silica, under an argon stream. These compositions may also comprise substances other than diluents, for example one or more lubricants such as magnesium stearate or talc, a coloring, a coating (sugar-coated tablet) or a glaze.

As liquid compositions for oral administration, there may be used pharmaceutically acceptable solutions, suspensions, emulsions, syrups and elixirs containing inert diluents such as water, ethanol, glycerol, vegetable oils or paraffin oil. These compositions may comprise substances other than diluents, for example wetting, sweetening, thickening, flavoring or stabilizing products.

The doses depend on the desired effect, the duration of the treatment and the route of administration used.

The invention is also related to the use of a polypeptide or a polynucleotide as described herein for the manufacture of a medicament for treatment of cancer and more particularly for treatment of breast cancer, kidney cancer and pancreatic cancer.

The present invention also provides methods for treating cancers including administering an effective amount of a polypeptide or a polynucleotide as described herein to a human or to a patient in need thereof. In a preferred embodiment, the invention relates to methods for treatment of breast cancer, kidney cancer and pancreatic cancer.

The present invention is also method for detecting a cancer in a patient comprising the following steps:
Measuring the level of expression of the PLA2R1 gene in a sample previously taken from a patient,
Comparing the expression level of the PLA2R1 gene with the expression level of the PLA2R1 gene in a control sample,
   wherein a decrease of the PLA2R1 expression level in the sample previously taken from the patient compared to the level of expression of the PLA2R1 gene in the control sample is significant of the presence of a cancer.

Another object of the present invention is a method for determining the prognosis of a cancer in a patient comprising the following steps:
c) Measuring the level of expression of the PLA2R1 gene in cancer cells from a tumour sample previously taken from said patient,
d) Classifying the cancer as having a poor prognosis if the PLA2R1 gene is under-expressed in said cancer cells.

The present invention also relates to methods for determining the prognosis of a cancer in a patient comprising the following steps:
a) Obtaining a tumour sample from said patient,
b) Measuring the level of expression of the PLA2R1 gene in cancer cells from said tumour sample,
c) Classifying the cancer as having a poor prognosis if the PLA2R1 gene is under-expressed in said cancer cells.

The present invention also relates to methods of determining the prognosis of a patient diagnosed with cancer.

“Cancer” refers to diseases in which a group of cells displays uncontrolled growth/division, invasion and sometimes metastasis.

The methods of the present invention preferably relate to breast cancer, kidney cancer, pancreatic cancer, colorectal cancer and melanoma. In preferred embodiments, the methods of the present invention relate to breast cancer, kidney cancer or pancreatic cancer and even more preferably to breast cancer and kidney cancer.

In a first embodiment, the present invention is related to a method for diagnosis/prognosis of cancer comprising determining the level of expression of PLA2R1 in a biological sample from a human subject.

Preferably, the methods of the present invention are in vitro methods. The methods of the present invention are carried out on a biological sample previously taken from a patient. Typically, the patient has been diagnosed with cancer or is suspected of suffering from cancer.

The term “sample” refers to any biological sample obtained/taken from a patient including a blood sample, a plasma sample, a tissue sample, a cell sample or a tumor sample. Typically, the sample contains cancer or tumors cells.

The term “diagnosis” refers both to diagnosis and prognosis of cancer.

The present invention further relates to a method for identifying a cancer and/or a tumor having or prone to develop an invasive or metastatic phenotype.

The terms “invasive” or “aggressive” refer to a cancer or to a quickly growing tumor having a tendency to extend beyond its boundaries into adjacent tissues.

The term “metastatic” refers to the spread of a tumor from the organ of origin to additional organs/distal sites in the patient.

A “prognosis” is the likely course and outcome of a disease. The prognosis may include the likelihood of complications of the cancer, of metastasis, of spread, probable outcome of the cancer, likelihood of recovery, overall survival rate and/or overall death rate. Preferably, it is the probability that a patient will recover or have a recurrence/relapse of the cancer. This information is useful to the patient but also to the physician in determining the most effective course of treatment. A determination of the likelihood for a cancer relapse or of the likelihood of metastasis can assist the physician in determining whether a more conservative or a more radical approach to therapy should be taken. Prognosis provides for selection and classification of patients who are predicted to benefit from a given therapeutic regimen.

The methods of the present invention provide prognosis for cancer after it has been diagnosed and/or during therapeutic treatment.

Under-expression or low expression levels of PLA2R1 in cancer cells are characteristic of tumors having a poor prognosis for disease-free survival (DFS) and/or a poor prognosis for overall survival (OS). Under-expression of this gene in cancer cells is statistically significantly correlated with increased disease recurrence and worse prognosis.

The present invention relates to a method for identifying a cancer and/or a tumor prone to recur and/or a cancer and/or a tumor having or prone to develop an invasive or metastatic phenotype. More specifically, the present invention relates to a method for identifying a cancer and/or a breast tumor prone to recur and/or a cancer and/or a tumor having or prone to develop an invasive or metastatic phenotype.

The term “DFS” refers to Disease Free Survival and is defined as the percentage of patients staying free of disease progression during a period of time. In this case, the Kaplan-Meier curve represents the x % of patients staying free of disease progression after y amount of time.

The DFS in a patient diagnosed with a cancer exhibiting under-expression or low expression levels of PLA2R1 in cancer cells, is reduced compared to a patient who has not an under-expression of PLA2R1 in cancer cells.

The term “OS” refers to Overall Survival and is defined as the percentage of patients who survive after diagnosis of a cancer. In this case, the Kaplan Meier curve represents the x % of patients who survived after y amount of time.

The OS in a patient diagnosed with a cancer exhibiting under-expression or low expression levels of PLA2R1 in cancer cells, is reduced compared to a patient who has not an under-expression of PLA2R1 in cancer cells.

The present invention is also method for diagnosing a cancer in a patient comprising the following steps:
Measuring the level of expression of the PLA2R1 gene in a sample previously taken from a patient,
Comparing the expression level of the PLA2R1 gene with the expression level of the PLA2R1 gene in a control sample,
   wherein a decrease of the PLA2R1 expression level in the sample previously taken from the patient compared to the level of expression of the PLA2R1 gene in the control sample is significant of an invasive, aggressive or metastatic cancer more particularly of a breast cancer, kidney cancer, pancreatic cancer colorectal cancer or melanoma.

In the methods of the present invention, the level of expression of the PLA2R1 gene is preferably compared to a control sample.

The control may be a “normal” control sample meaning a non-tumor cell control sample or a non-cancerous cell control sample. The control sample can be an autologous control sample obtained from the patient. In that case the sample is obtained from the same patient from which the sample to be evaluated is obtained. The control sample is preferably from the same cell type or from the same organ. The control sample can be a normal control sample obtained from an individual who does not have cancer.

The normal control sample can also be a standard sample that contains the same amount of PLA2R1 that is normally found in biological samples.

In some embodiments, the control sample is the median level of expression of the PLA2R1 gene observed in a healthy population.

In other embodiments, the control sample is the median level of expression of PLA2R1 in samples taken from patients suffering from a cancer and more particularly from breast cancer, kidney cancer, pancreatic cancer colorectal cancer or melanoma.

The term “expression” may refer to measuring the level of transcription and/or the level of translation of the gene.

Preferably, the methods of the present invention comprise measuring the level of expression of the PLA2R1 gene in a sample by quantification of the mRNA of the PLA2R1 gene.

In other embodiments, the methods of the present invention comprise measuring the level of expression of the PLA2R1 gene in a sample by quantification of the polypeptide(s) encoded by the PLA2R1 gene.

Expression levels of the PLA2R1 gene are determined by any of a variety of methods known to the skilled person.

### FIGURES

FIG. 1: A/ mRNA were extracted for each cell line. qRT-PCR were performed and relative PLA2R1 mRNA levels were calculated using ACTB, PGK1 as housekeeping genes (*P<0, 05; **P<0, 01; ***P<0, 001).

B/ mRNA were from 18 normal kidney and 18 tumoral kidney samples were prepared. qRT-PCR were performed and relative PLA2R1 mRNA levels were calculated using ACTB housekeeping gene.

C/ Cells were infected by a control or a PLA2R1 encoding vector and puromycin selected. Cell extracts were prepared and analyzed by immunoblot for PLA2R1 expression. TUB (tubulin) was used as a loading control.

D/ Cells seeded at the same density were infected by a control or a PLA2R1 encoding vector, in conditions were all cells were infected. Puromycin was added every day to maintain PLA2R1 expression. Ten days later, cells were PFA-fixed and stained with crystal violet.

E/ Cells extracts were prepared as in (B) and analyzed by immunoblot for the presence of cleaved caspase-3 (c-CASP3). Actin (ACTB) was used as a loading control.

FIG. 2: Cells were seeded at the same density, infected by a retrovirus coding for no protein (control), WT PLA2R1 or soluble deletion mutants of PLA2R1, in conditions where all cells were infected. Puromycin was added every day to maintain PLA2R1 expression.

A/ The effect of control, WT PLA2R1 and CysR-CTLD8 PLA2R1 were assayed in cama-1, BT-20 and MDAMB-453 breast cancer cells for the induction of cell death.

B/ The effect over cell death of CysR-CTLD8, CysR-CTLD6, CysR-CTLD3 and CysR-FNII soluble forms of PLA2R1 were assayed in Cama-1 breast cancer cells.

FIG. 3: Under-expression of PLA2R1 in cancer cells correlates with an increased risk of developing metastasis.

FIG. 4: Under-expression of PLA2R1 in cancer cells correlates with decreased Overall Survival (OS).

### EXAMPLES

### Materials and Methods

### Cell Culture

Human cancer cell lines (ATCC) were cultured in the following media: DMEM (Invitrogen) for MDA-MB 453, MDA-MB-157, MDA-MB-231, MDA-MB-361, MDA-MB-436, Hs-578T, MCF-7, Cal-51, Caki-2, 786-O, RPMI (Invitrogen) for RCC4, RCC10, MEM (Invitrogen) for Cama-1, BT-20, ACHN and Mc Coy's (Invitrogen) for SK-Br3 cells. All medium were supplemented with 10% FBS (Lonza), 1% penicillin/streptomycin (Invitrogen), 0, 36% gentamycin (Invitrogen). For Cama-1 and ACHN cells lines, 1% Non essential Amino-Acid (Invitrogen) was added. Virus producing GP293 cells (Clontech) were cultured in DMEM media (Invitrogen) supplemented with 10% FBS (Lonza), 1% penicillin/streptomycin (Invitrogen) and 0, 36% gentamycin (Invitrogen).

### Vectors Construction

Wild-type membrane-bound (GenBank NM 007366) and soluble deletion mutants of human PLA2R1 were generated by PCR. Each construct was first ligated into the pGEMTeasy vector (Promega), fully sequenced and subsequently subcloned in the pLPCX retroviral vector (Clontech) using XhoI/NotI restriction sites. The PLA2R1 WT (amino acids (aa) 1-1463) and soluble deletion mutants of PLA2R1, namely CysR-FNIIs (aa 1-222), CysR-CTLD3 (aa 1-645) CysR-CTLD6 (aa 1-1098) and CysR-CTLD8 (aa 1-1322) were obtained by using the same forward primer with different reverse primers whose sequences are: Forward, 5′-TACTCGAGCCACCATGCTGCTGTCGCCGTCGCTG-3′; WT Reverse, 5′-TAGCGGCCGCTTATTGGTCACTCTTCTCAAGATC-3′; FNII reverse, 5′-TAGCGGCCGCTTAGGGATCAGGGCAAAATCCCC-3; CTLD3 reverse, 5′-TAGCGGCCGCTTACTGCTTGCACAAGGACATTGC-3′; CTLD6 reverse, 5′-TAGCGGCCGCTTATTTTTCACAAACAAACCCATAGCCTTC-3; CTLD8 reverse, 5′-TAGCGGCCGCTCACTTACTGTTACCATCAAATTGAGCATTCAA-3′. PCR conditions were 98° C. for 1 minute, followed by 30 cycles of 98° C. for 10 seconds, 60° C. for 30 seconds, 72° C. for 1-5 minutes. This was followed by a final extension of 72° C. for 10 minutes.

### Transfection and Infection

GP293 cells were transfected using PEI reagents according to manufacturer's recommendations (Euromedex). Two days after transfection, viral supernatant mixed with fresh media (1/2) and polybrene (final concentration at 8 ug/mL) was used to infect target cells. One day post infection, cells were selected using puromycin at the final concentration of 0.75-1.5 μg/ml depending on the cell type.

### Antibodies and Immunoblot

Cell lysates were prepared in ice cold Giordano buffer (50 mM Tris.HCl, pH 7.4, 250 mM NaCl, 0.2% Triton X-100, 5 mM EDTA) supplemented with protease and eroxidise inhibitors (Roche). Lysates were clarified by centrifugation at 14,000 rpm for 30 min at 4° C. Protein concentrations were measured using Bradford protein assay (Biorad #500-0006). For immunoblot analysis, cell extracts were separated by SDS-PAGE gel electrophoresis under non reducing conditions and proteins were transferred onto nitrocellulose membrane. The following primary antibodies were used: anti-PLA2R1 (HPA012657, Atlas), anti-caspase3 (ab32042, Abcam), anti-tubulin (T6199, Sigma) and anti-actin (A5316, Sigma). After incubation with primary antibodies, blots were washed, incubated with a secondary antibody coupled to peroxidase, washed again and antigen-antibody complexes were detected using ECL (Amersham).

### Bioinformatics Analysis

The analysis was performed by interrogating the oncomine public database.

### RNA Extraction, Retro-Transcription and Quantitative PCR

Normal and tumoral human kidney tissues were provided by the tumorotheque HEH. Total RNA extraction was performed using a phenol-chloroform method using TriReagent (Sigma-Aldrich, Saint Louis, Mo., USA). PhaseLockGel tubes (Eppendorf, Hamburg, Germany) were used for phase separation. The synthesis of cDNA was performed from 3 ug of total RNA using the First-Strand cDNA Synthesis Kit (GE Healthcare, Chalfont St Giles, UK). The RT reaction was diluted 1/60 and used as cDNA template for qPCR analysis. TaqMan quantitative PCR analysis was carried out on a LightCycler 2.0 System (Roche Applied Science). PCR mixtures contained LightCycler TaqMan mix, 200 nM primers and 1.67 μl of cDNA template in a 6.67 μl reaction volume. Housekeeping genes (ACTB, PGK1), used for normalization of target mRNA expression in each sample type, were selected by systematic geNorm analysis as previously described (Vandesompele et al., 2002). Real-time PCR intron-spanning assays were designed using the ProbeFinder software (Roche Applied Science).

### Colony Formation Assays

Colony formation assays were carried out in 12-well plates with 100000 cells per well for MDAMB-453 and Cama-1 cells, and 25000 cells plated per well for BT-20 and MD-AMB-231 cells. Ten days later, cells were washed with PBS, fixed with 4% paraformaldehyde and stained with 0.05% Crystal violet (Sigma-Aldrich).

### Results

### PLA2R1 Expression is Decreased in Breast and Kidney Cancers

To analyze the expression levels of PLA2R1 in various types of cancer, we performed a bio-informatic analysis of public databases.

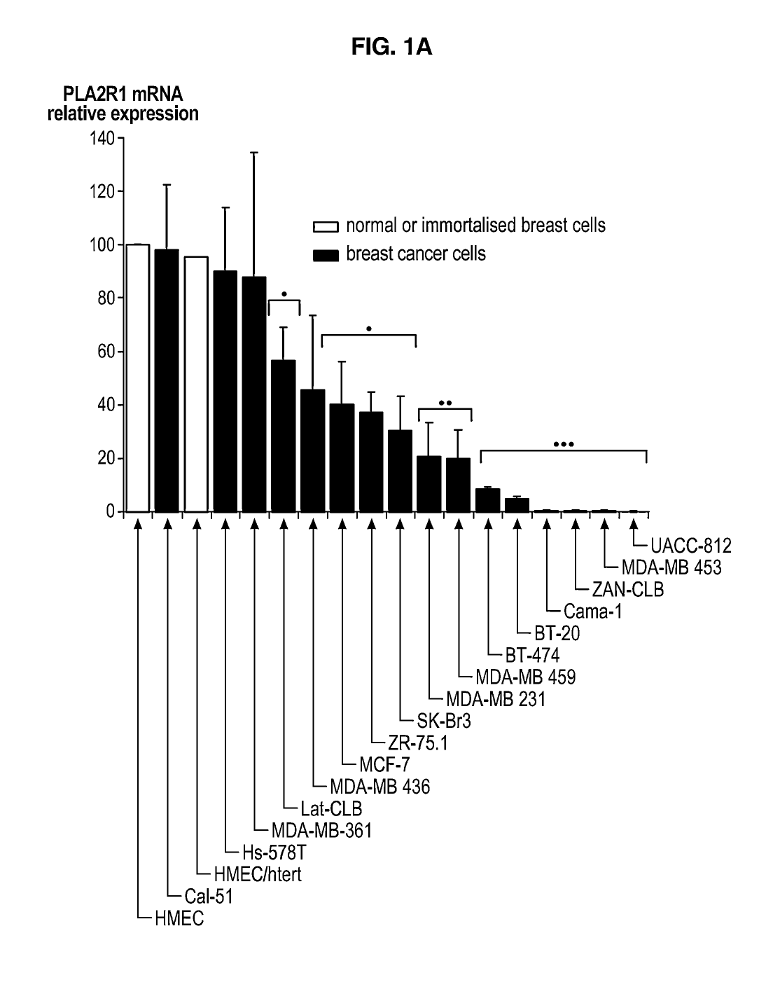

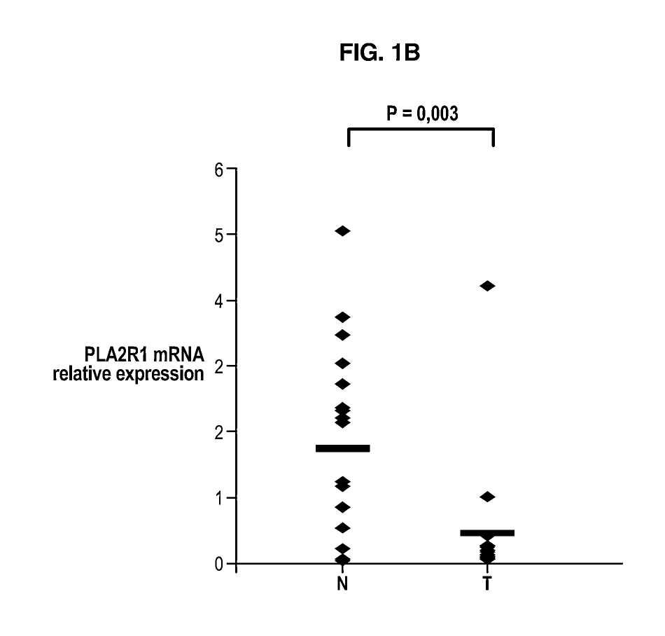

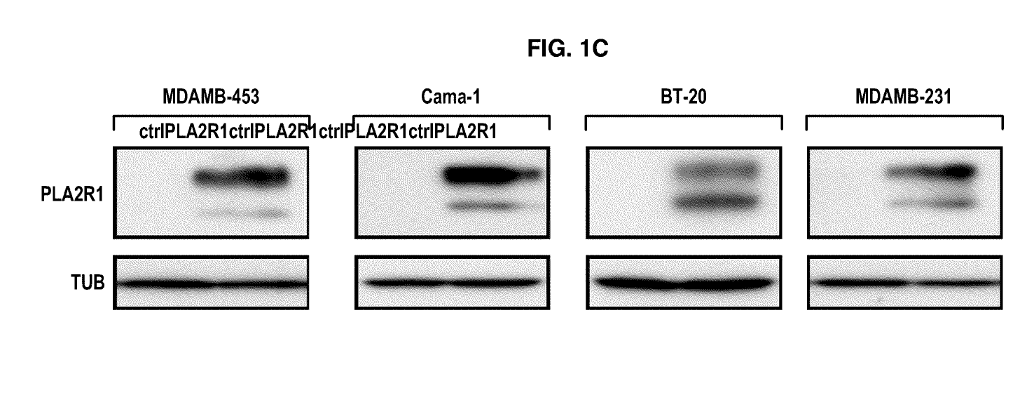

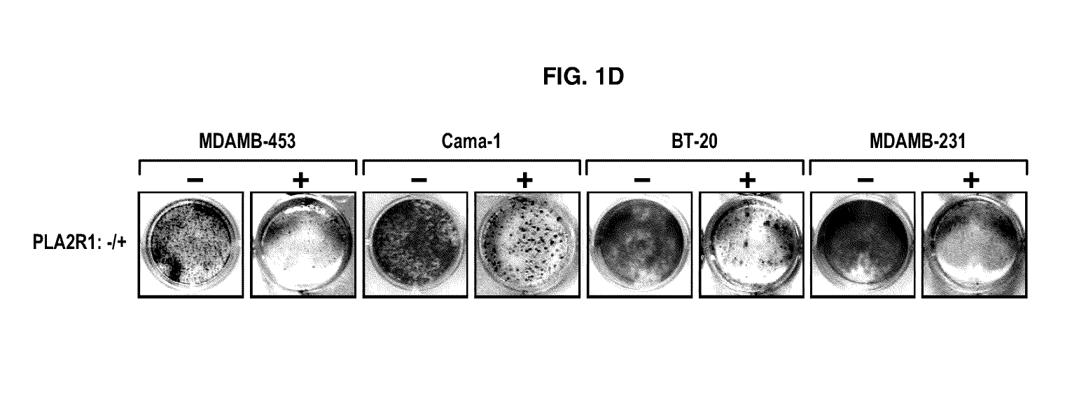

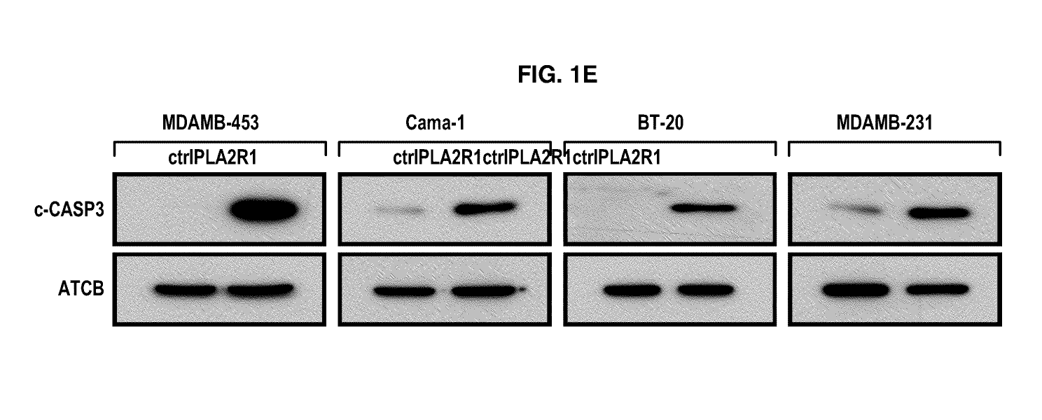

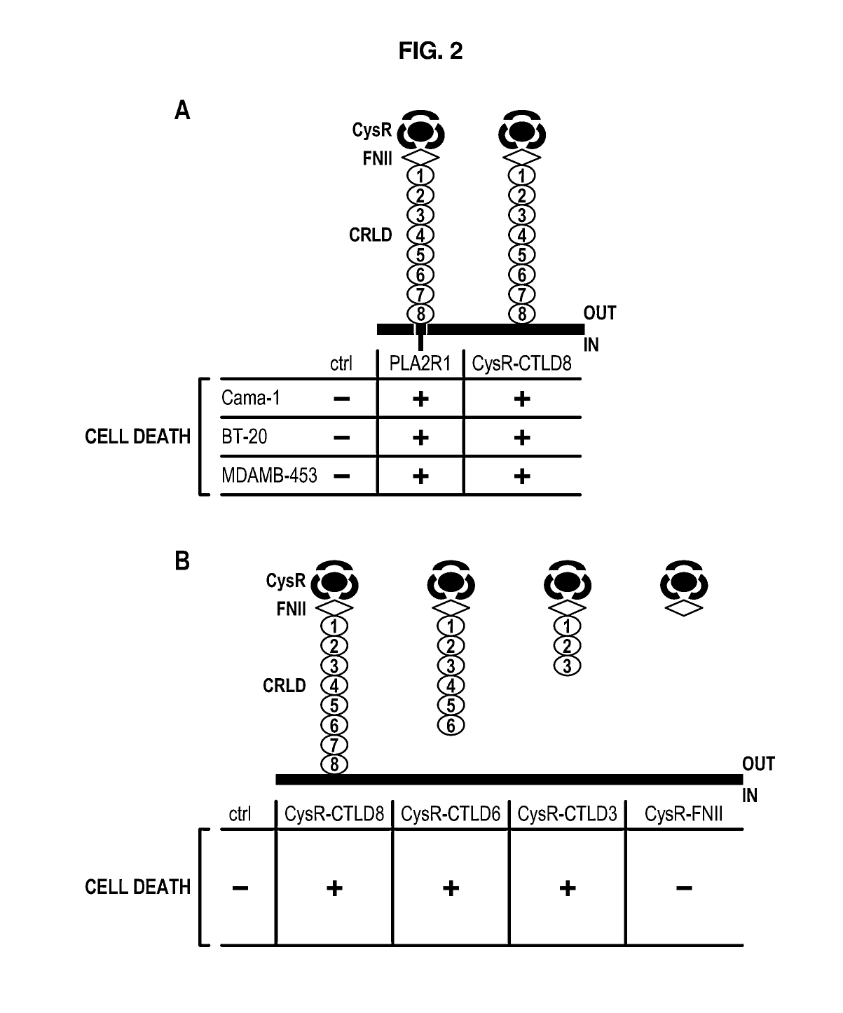

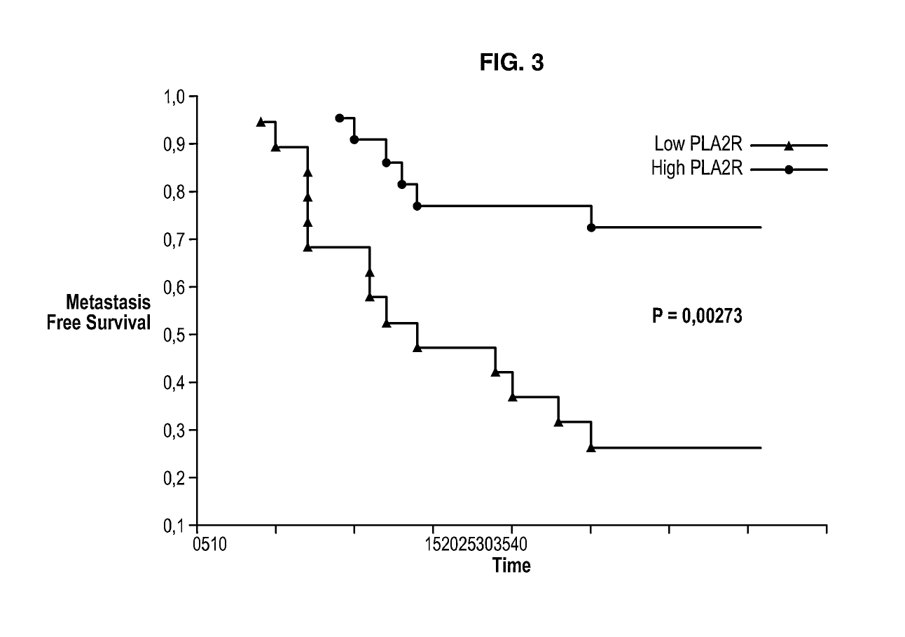

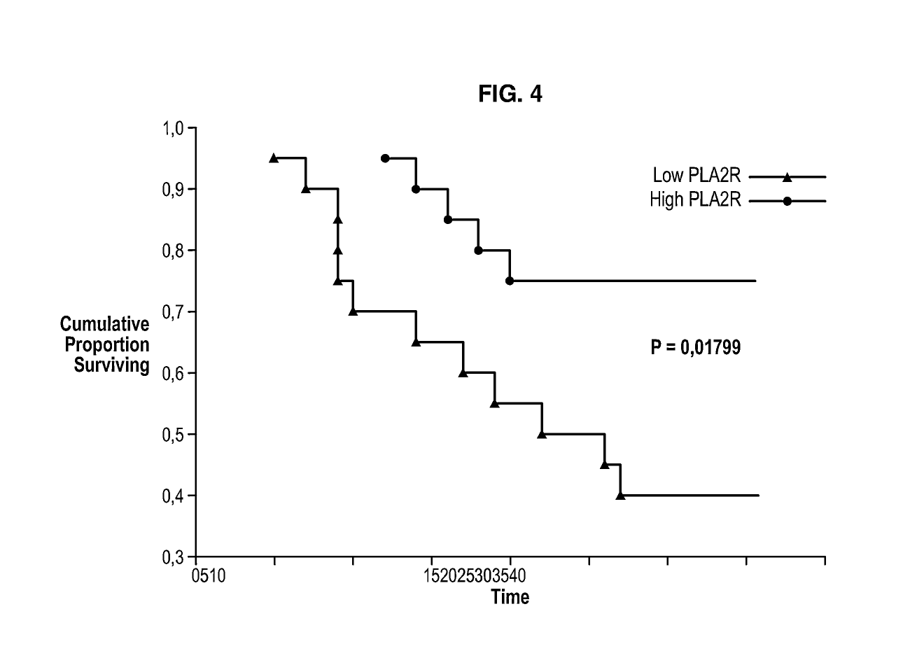

The oncomine public database was interrogated for a differential level of PLA2R1 mRNA levels between cancer and normal counterpart in 239 independent set of microarrays analyses. Analysis were performed according to the following criteria: 1/fold change of PLA2R1 mRNA levels between cancer and normal counterpart, 2/reproducibility in the differentials between independent studies from the same type of cancer, 3/fold-change ranking of PLA2R1 in the differentially expressed genes between cancer and normal counterpart and 4/the p-value. According to these criteria, PLA2R1 mRNA levels were found to significantly decrease in breast and kidney cancers when compared to normal counterpart (Table 1 and 2). This bio-informatics analysis was further confirmed: 1/by analyzing PLA2R1 mRNA levels in normal or immortalized human breast cells vs human breast cancer cells. Indeed, 13 out of 15 breast cancer cell lines examined displayed a significant decrease in PLA2R1 mRNA levels when compared to normal breast cells (FIG. 1A) 2/by analyzing PLA2R1 mRNA levels in normal vs tumoral human kidney samples (FIG. 1B).

Thus a decrease in PLA2R1 expression constitutes a new biomarker of breast and kidney cancers.

TABLE 1
Summary of PLA2R1 expression in breast tumors versus normal samples. Six out
of 8 studies indicate significant (p < 0.05) down-regulation of PLA2R1 in breast tumors

| | | | | Under-expression |
|---|---|---|---|---|
| Name of the study | Tissues compared | Fold changes | P value | Gene Rank |
| Zhao Breast | Invasive Ductal Breast | −2.52 | 1.30E−15 | in top 1% |
| | Carcinoma vs. Normal | | | |
| | Lobular Breast | −1.81 | 1.45E−6 | In top 1% |
| | Carcinoma vs. Normal | | | |
| Richardson Breast | Ductal Breast Carcinoma | −3.684 | 2.13E−8 | in top 3% |
| 2 | vs. Normal | | | |
| Sorlie Breast | Lobular Breast | −1.757 | 0.036 | in top 7% |
| | Carcinoma vs. Normal | | | |
| | Ductal Breast Carcinoma | −2.484 | 0.009 | in top 10% |
| | vs. Normal | | | |
| Sorlie Breast 2 | Lobular Breast | −1.789 | 0.026 | in top 9% |
| | Carcinoma vs. Normal | | | |
| | Ductal Breast Carcinoma | −2.498 | 0.008 | in top 11% |
| | vs. Normal | | | |
| Perou Breast | Ductal Breast Carcinoma | −2.411 | 0.043 | in top 15% |
| | vs. Normal | | | |
| Turashvili Breast | Invasive Ductal Breast | −2.701 | 0.019 | in top 6% |
| | Carcinoma vs. Normal | | | |

TABLE 2
Summary of PLA2R1 expression in kidney tumors versus normal samples. Six out
of 6 studies indicate significant (p < 0.05) down-regulation of PLA2R1 in kidney tumors.

| | | | | Under-expression |
|---|---|---|---|---|
| Name of the study | Tissues compared | Fold changes | P value | Gene Rank |
| Yusenko Renal | Chromophobe Renal Cell | −76.573 | 4.14^{E}−5 | in top 1% |
| | Carcinoma vs. Normal | | | |
| | Papillary Renal Cell | −2.111 | 2.94^{E}−8 | in top 1% |
| | Carcinoma vs. Normal | | | |
| | Clear Cell Renal Cell | −13.649 | 2.46^{E}−8 | in top 1% |
| | Carcinoma vs. Normal | | | |
| Higgins Renal | Granular Renal Cell | −2.714 | 0.031 | in top 6% |
| | Carcinoma vs. Normal | | | |
| | Chromophobe Renal Cell | −3.081 | 0.031 | in top 9% |
| | Carcinoma vs. Normal | | | |
| | Papillary Renal Cell | −3.202 | 0.028 | in top 13% |
| | Carcinoma vs. Normal | | | |
| | Clear Cell Renal Cell | −2.612 | 0.042 | in top 19% |
| | Carcinoma vs. Normal | | | |
| Cutcliffe Renal | Renal Wilms Tumor vs. | −4.639 | 4.11^{E}−4 | in top 4% |
| | Normal | | | |
| | Clear Cell Sarcoma of | −6.910 | 1.16^{E}−4 | in top 4% |
| | the Kidney vs. Normal | | | |
| Lenburg Renal | Clear Cell Renal Cell | −2.032 | 1.83^{E}−4 | in top 4% |
| | Carcinoma vs. Normal | | | |
| Beroukhim Renal | Non-Hereditary Clear | −1.540 | 4.38^{E}−4 | in top 13% |
| | Cell Renal Cell | | | |
| | Carcinoma vs. Normal | | | |
| | Hereditary Clear Cell | −1.576 | 2.96^{E}−4 | in top 20% |
| | Renal Cell Carcinoma vs. | | | |
| | Normal | | | |
| Gumz Renal | Clear Cell Renal Cell | −3.745 | 0.006 | in top 19% |
| | Carcinoma vs. Normal | | | |

### PLA2R1 Expression Induces Cancer Cell Death

In order to examine the effect of restoring PLA2R1, we have constructed a retroviral vector encoding the full length PLA2R1. PLA2R1 non-expressing cells such as MDAMB-453 and cama-1 breast cancer cells (FIG. 1A) were infected by a retroviral vector encoding PLA2R1. Three days post-infection, the cells clearly express PLA2R1 according to the western blot analysis (FIG. 1C). Strikingly, PLA2R1 constitutive expression induced a strong growth arrest (FIG. 1D) that was due to apoptotic cell death based on the appearance of active cleaved caspase-3 (FIG. 1E). Interestingly, cells expressing low levels of PLA2R1 mRNA (BT-20 and MDAMB-231, FIG. 1A) were also dying by apoptotic cell death in response to PLA2R1 ectopic expression (FIG. 1C-E). In fact, most of the breast cancer cells tested (10 out of 11) died when PLA2R1 was constitutively expressed, without any link with their endogenous mRNA PLA2R1 levels (Table 3). To determine whether the toxic effect of PLA2R1 over-expression is specific to breast cancer cells or more general, we investigated the effect of PLA2R1 ectopic expression in kidney cancer, pancreatic cancer, colorectal cancer and melanoma cells. Five kidney, 2 pancreatic, 1 colorectal cancer and 2 melanoma cell lines were transduced to express PLA2R1 and all of them died in response to PLA2R1 (Table 3). Thus, PLA2R1 constitutive expression induced cell death in almost all (20/21) the cancer cell lines tested.

To determine which PLA2R1 domains are involved in cell death-induced by PLA2R1 ectopic expression, we generated a set of shorter PLA2R1 proteins. A mutant deleted from the intra-cellular domain and of the transmembrane domain (CysR-CTLD8 mutant) was still able to induce apoptotic cell death (FIG. 2A). So far, the CysR-CTLD3 mutant, encompassing the cysteine rich domain, the fibronectin-like domain and CTLDs 1 to 3, is the shortest mutant that is still capable of inducing cell death (FIG. 2B).

Thus, full-length PLA2R1 and various soluble variants of PLA2R1 are capable of inducing tumor cell death.

TABLE 3
Sensitivity (cell death) or resistance (no cell death) to PLA2R1
expression of every human cancer cell lines tested is presented.

| Name of the cell | Sensitive to | Resistant to | |
|---|---|---|---|
| lines | PLA2R1 | PLA2R1 | Tumor origin |
| MDAMB-453 | X | | Breast |
| Cama-1 | X | | Breast |
| BT-20 | X | | Breast |
| MDAMB-157 | X | | Breast |
| MCF-7 | X | | Breast |
| Cal-51 | X | | Breast |
| MDAMB-436 | X | | Breast |
| MDAMB-231 | X | | Breast |
| MDAMB-361 | X | | Breast |
| Hs-578T | X | | Breast |
| SKBR-3 | | X | Breast |
| Caki-2 | X | | Kidney |
| ACHN | X | | Kidney |
| 786-0 | X | | Kidney |
| RCC4 | X | | Kidney |
| RCC10 | X | | Kidney |
| MIA PaCa-2 | X | | Pancreas |
| Colo 357 | X | | Pancreas |
| A375 | X | | melanoma |
| SK-Mel | X | | melanoma |
| RPMI 7951 | X | | melanoma |
| Lovo | X | | colorectal |
| HT-29 | X | | colorectal |

In conclusion, we have described a new tumor-suppressive-like role of PLA2R1 in cancer. Since PLA2R1 levels decrease in both breast and kidney cancers, PLA2R1 can be useful as a biomarker for these cancer types. In addition, since over-expression of PLA2R1 (full-length or soluble variants) to almost all of the breast, kidney and pancreatic cancer cells induces cell death, PLA2R1 or these shorter mutants can be useful as anti-tumoral biomolecules.

### PLA2R1 Expression Decrease Correlates to an Increase Risk of Developing Metastasis and of Dying

The expression of PLA2R1 decreases in breast and kidney cancer cells. To know whether this decrease might be associated with a clinical behavior, we examined the expression of PLA2R1 on breast primary tumors coming from 20 patients that have developed some metastasis and have died in less than 3 years and from 20 patients that have not developed any metastasis and have not died at 3 years. PLA2R1 mRNA levels were determined by RT-qPCR. A first group containing the half of the samples expressing the higher levels of PLA2R1 and second group containing the half of the samples expressing the lower levels of PLA2R1 was constituted. Survival and metastasis free survival were analyzed in the 2 groups by Kaplan-Meier curves. Interestingly, low level of PLA2R1 was associated with an increase risk of developing metastasis and of dying (see FIGS. 3 and 4).

### REFERENCES

Augert, A., Payre, C., de Launoit, Y., Gil, J., Lambeau, G., and Bernard, D. (2009). The M-type receptor PLA2R regulates senescence through the p53 pathway. EMBO Rep 10, 271-277.
Braig, M., Lee, S., Loddenkemper, C., Rudolph, C., Peters, A. H., Schlegelberger, B., Stein, H., Dorken, B., Jenuwein, T., and Schmitt, C. A. (2005). Oncogene-induced senescence as an initial barrier in lymphoma development. Nature 436, 660-665.
Campisi, J. (2011). Cellular senescence: putting the paradoxes in perspective. Curr. Opin. Genet. Dev. 21, 107-112.
Chen, Z., Trotman, L C, Shaffer, D., Lin, H. K., Dotan, Z. A., Niki, M., Koutcher, J. A., Scher, H. I., Ludwig, T., Gerald, W., Cordon-Cardo, C., and Paolo Pandolfi, P. (2005). Crucial role of p53-dependent cellular senescence in suppression of Pten-deficient tumorigenesis. Nature 436, 725-730.
Collado, M., Gil, J., Efeyan, A., Guerra, C., Schuhmacher, A. J., Barradas, M., Benguria, A., Zaballos, A., Flores, J. M., Barbacid, M., Beach, D., and Serrano, M. (2005). Tumour biology: Senescence in premalignant tumours. Nature 436, 642.
Engelholm, L. H., Ingvarsen, S., Jurgensen, H. J., Hillig, T., Madsen, D. H., Nielsen, B. S., and Behrendt, N. (2009). The collagen receptor uPARAP/Endo180. Front Biosci. 14, 2103-2114.
Ewald, J. A., Desotelle, J. A., Wilding, G., and Jarrard, D. F. (2010). Therapy-induced senescence in cancer. J. Natl. Cancer Inst. 102, 1536-1546.
Hanahan, D. and Weinberg, R. A. (2000). The hallmarks of cancer. Cell 100, 57-70.
Lambeau, G. and Lazdunski, M. (1999). Receptors for a growing family of secreted phospholipases A2. Trends Pharmacol. Sci. 20, 162-170.
Murakami, M., Taketomi, Y., Girard, C., Yamamoto, K., and Lambeau, G. (2010). Emerging roles of secreted phospholipase A2 enzymes: Lessons from transgenic and knockout mice. Biochimie 92, 561-582.
Rodier, F. and Campisi, J. (2011). Four faces of cellular senescence. J. Cell Biol. 192, 547-556.
Shrimpton, R. E., Butler, M., Morel, A. S., Eren, E., Hue, S. S., and Ritter, M. A. (2009). CD205 (DEC-205): a recognition receptor for apoptotic and necrotic self Mol. Immunol. 46, 1229-1239.
Taylor, P. R., Gordon, S., and Martinez-Pomares, L. (2005). The mannose receptor-linking homeostasis and immunity through sugar recognition. Trends Immunol. 26, 104-110.

1. A method for treating cancer in a patient in need thereof, comprising administering to said patient an effective amount of a composition comprising a polynucleotide selected from the group consisting of:
The polynucleotide of SEQ ID NO. 1 or a variant thereof having at least 95% identity with the sequence of SEQ ID No. 1;
A polynucleotide encoding the human PLA2R1 polypeptide of SEQ ID NO. 2 or encoding a variant thereof having at least 95% identity with the sequence of SEQ ID No. 2;
A polynucleotide from position 1 to position 4191 of SEQ ID NO.1 encoding the extracellular domain of human PLA2R1 or a variant thereof having at least 95% identity with the polynucleotide from position 1 to position 4191 of SEQ ID NO.1;
A fragment of the polynucleotide from position 1 to position 4191 of SEQ ID NO.1 encoding a fragment of the extracellular domain of PLA2R1 or a variant thereof having at least 95% identity with a fragment of the polynucleotide from position 1 to position 4191 of SEQ ID NO.1; and
A polynucleotide encoding a polypeptide of SEQ ID NO. 4, 6 or 8.

2. The method of claim 1 wherein the fragment of the polynucleotide from position 1 to position 4191 of SEQ ID NO.1 encodes at least the Cys-rich domain, the fibronectin type II domain and a C-type lectin domain of human PLA2R1.

3. The method of claim 1 wherein the fragment of the polynucleotide from position 1 to position 4191 of SEQ ID NO.1 is selected from the group consisting of the polynucleotides of SEQ ID NO. 3, SEQ ID NO. 5, SEQ ID NO. 7 and variants thereof having at least 95% identity with the polynucleotide of SEQ ID NO. 3, SEQ ID NO.5 or SEQ ID NO.7.

4. The method of claim 1 wherein said polynucleotide is inserted in a viral vector.

5. The method of claim 1 wherein said polynucleotide is incorporated in a delivery vehicle.

6. A method for treating cancer in a patient in need thereof, comprising administering to said patient an effective amount of a composition comprising a polypeptide selected from the group consisting of:
The Human PLA2R1 polypeptide depicted in SEQ ID NO. 2 or a variant thereof having at least 95% identity with the polypeptide of SEQ ID No. 2;
The extracellular domain of human PLA2R1 polypeptide from position 21 to position 1397 of SEQ ID NO.2 or a variant thereof having at least 95% identity with the human PLA2R1 polypeptide from position 21 to position 1397 of SEQ ID NO.2; and
A fragment of the extracellular domain of human PLA2R1 polypeptide from position 21 to position 1397 of SEQ ID NO.2 or a variant thereof having at least 95% identity with a fragment the human PLA2R1 polypeptide from position 21 to position 1397 of SEQ ID NO.2.

7. The method of claim 6 wherein the fragment of the extracellular domain of human PLA2R1 polypeptide from position 21 to position 1397 of SEQ ID NO.2 comprises at least the Cys-rich domain, the fibronectin type II domain and a C-type lectin domain of human PLA2R1.

8. The method of claim 6 wherein the fragment of the extracellular domain of human PLA2R1 polypeptide from position 21 to position 1397 of SEQ ID NO.2 is selected from the group consisting of the polypeptide from position 21 to position 1322 of SEQ ID NO. 4, the polypeptide from position 21 to position 1098 of SEQ ID NO. 6, the polypeptide from position 21 to position 645 of SEQ ID NO. 7 and variants thereof having at least 95% identity with the polypeptide from position 21 to position 1322 of SEQ ID NO. 4, at least 95% identity with the polypeptide from position 21 to position 1098 of SEQ ID NO. 6 or at least 95% identity with the polypeptide from position 21 to position 645 of SEQ ID NO. 8.

9. The method of claim 1 wherein the cancer is selected from the group consisting of breast cancer, pancreatic cancer, kidney cancer, colorectal cancer and melanoma.

10. A polypeptide encoding a soluble extracellular fragment of human PLA2R1 comprising the polypeptide of SEQ ID NOs. 4, 6, 8 or variants thereof having at least 95% identity with the polypeptide of SEQ ID NO. 4, at least 95% identity with the polypeptide of SEQ ID NO. 6 or at least 95% identity with the polypeptide of SEQ ID NO. 8.

11. (canceled)

12. Polynucleotide encoding a polypeptide according to claim 10.

13. (canceled)

14. (canceled)

15. (canceled)

16. The method of claim 6 wherein the cancer is selected from the group consisting of breast cancer, pancreatic cancer, kidney cancer, colorectal cancer and melanoma.


SEQUENCE LISTING (16 sequences)

SEQ ID NO: 1 - DNA, 4392 bp, Homo sapiens
  CDS (1)..(4389)
atgctgctgtcgccgtcgctgctgctgctgctgctgctgggggcgccgcggggctgcgccgagggtgtggcggcggcgcttacccccgagcggctcctggagtggcaggataaaggaatatttgttatccaaagtgagagtctcaagaaatgcattcaagcaggtaaatcggttctgaccctggagaactgcaagcaagcaaacaagcacatgctgtggaaatgggtttcaaaccatggcctctttaacataggaggcagcggttgcctgggcctgaatttctccgccccagagcagccattaagcttatatgaatgtgactccaccctcgtttccttacggtggcgctgtaacaggaagatgatcacaggcccgctgcagtactctgtccaggtggcgcatgacaacacagtggtggcctcacggaagtatattcataagtggatttcttatgggtcaggtggtggagacatttgtgaatatctacacaaagatttgcatacaatcaaagggaacacccacgggatgccgtgtatgtttcccttccagtataaccatcagtggcatcatgaatgtacccgtgaaggtcgggaagatgacttactgtggtgtgccacgacaagccgttatgaaagagatgaaaagtggggattttgccctgatcccacctctgcagaagtaggttgtgatactatttgggagaaggacctcaattcacacatttgctaccagttcaacctgctttcatctctctcttggagtgaggcacattcttcatgccagatgcaaggaggtacgctgttaagtattacagatgaaactgaagaaaatttcataagggagcacatgagcagtaaaacagtggaggtgtgggtgggcctcaatcagctggatgaagacgctggctggcagtggtctgatggaacgccgctcaactatctgaattggagcccagaggtaaattttgagccatttgttgaagatcactgtggaacatttagttcatttatgccaagtgcctggaggagtcgggattgtgagtccaccttgccatatatatgtaaaaaatatctaaaccacattgatcatgaaatagttgaaaaagatgcgtggaaatattatgctacccactgtgagcctggctggaatccctacaatcgtaattgctacaaacttcagaaagaagaaaagacctggcatgaggctctgcgttcttgtcaggctgataacagtgcattaatagacataacctcattagcagaggtggagtttcttgtaaccctccttggagatgaaaatgcatcagaaacatggattggtttgagcagcaataaaattccagtttcctttgaatggtctaatgactcttcagtcatctttactaattggcacacacttgagccccacatttttccaaatagaagccagctgtgtgtctcagcagagcagtctgagggacactggaaagtcaaaaattgtgaagaaagacttttttacatttgtaaaaaagcaggccatgtcctctctgatgctgaatcaggatgtcaagagggatgggagagacatggtggattctgttacaaaattgacacagtccttcgaagctttgaccaagcttccagcggttattactgtcctcctgcacttgtaaccattacaaacaggtttgaacaggcttttattaccagtttgatcagtagtgtggtaaaaatgaaggacagttatttttggatagctcttcaggaccaaaatgatacgggagaatacacttggaagccagtagggcagaaacccgagccggtgcagtacacacactggaacacacaccagccgcgctacagtggtggctgtgttgccatgcgaggaaggcatccacttggtcgctgggaagtgaagcactgtcggcactttaaggcaatgtccttgtgcaagcagccagttgaaaatcaggaaaaagcagagtatgaagagagatggccctttcacccctgctatttggactgggagtcagagcctggtctggccagttgcttcaaggtatttcatagtgaaaaagttctgatgaaaagaacatggagagaagctgaagcattttgcgaagaatttggagctcatcttgcaagctttgcccatattgaggaagagaattttgtgaatgagctcttacattcaaaatttaattggacagaagaaaggcagttctggattggatttaataaaagaaacccactgaatgccggctcatgggagtggtctgatagaactcctgttgtctcttcgtttttagacaacacttattttggagaagatgcaagaaactgtgctgtttataaggcaaacaaaacattgctgcccttacactgtggttccaaacgtgaatggatatgcaaaatcccaagagatgtgaaacccaagattccgttctggtaccagtacgatgtaccctggctcttttatcaggatgcagaatacctttttcatacctttgcctcagaatggttgaactttgagtttgtctgtagctggctgcacagtgatcttctcacaattcattctgcacatgagcaagaattcatccacagcaaaataaaagcgctatcaaagtatggtgcaagttggtggattggacttcaagaagaaagagccaatgatgaatttcgctggagagatggaacaccagtgatataccagaactgggacacaggaagagaaagaactgtgaataatcagagccagagatgtggctttatttcttctataacaggactctggggtagtgaagagtgttcagtttctatgcctagtatctgtaagcgaaaaaaggtttggctcatagagaaaaagaaagatacaccaaaacaacatggaacgtgtcccaaaggatggctatattttaactataagtgccttctgctgaatatccccaaagacccaagcagttggaagaactggacgcatgctcaacatttctgtgctgaagaaggggggaccctggtcgccattgaaagtgaggtggagcaagctttcattactatgaatctttttggccagaccaccagtgtgtggataggtttacaaaatgatgattatgaaacatggctaaatggaaagcctgtggtatattctaactggtctccatttgatataataaatattccaagtcacaataccactgaagttcagaaacacattcctctctgtgccttactctcaagtaatcctaattttcatttcactggaaaatggtattttgaagactgtggaaaggaaggctatgggtttgtttgtgaaaaaatgcaagatacttctggacacggtgtaaatacatctgatatgtatccaatgcccaataccttagaatatggaaacagaacttacaaaataattaatgcaaatatgacttggtatgcagcaataaaaacctgcctgatgcacaaagcacaactggtcagcatcacagaccagtatcaccagtccttcctcactgttgtcctcaaccggctaggatatgcccactggattggactgttcaccacagataatggtcttaattttgactggtctgatggcaccaaatcttctttcactttttggaaagatgaggagtcctccctccttggtgactgcgtttttgccgacagcaacggacgctggcatagcacagcctgcgagtcatttctgcaaggtgccatttgtcatgtgccacctgaaacaagacaatctgaacacccagagttgtgctcagaaacatctattccctggataaaatttaaaagtaattgctacagtttttctacagtcctagacagtatgagttttgaggctgctcatgaattttgcaaaaaggaaggttctaatcttttaacaatcaaggatgaggctgaaaatgcatttctcctagaagagctgtttgcttttggttcttctgtccagatggtttggttgaatgctcaatttgatggtaacaatgaaaccataaagtggtttgatggaactcccacagaccagtcaaactggggcattcggaagccagacacagactacttcaagccccatcattgtgttgccttgaggatccctgaaggattatggcagctatccccgtgtcaagaaaaaaaaggctttatatgtaaaatggaggcagatattcacactgcagaggcgctgccagaaaaaggaccaagtcacagcatcattcctcttgcggttgtactgacactgatagtcattgtggccatttgcacactttccttctgcatatacaagcataacggtggcttcttcaggagacttgcagggtttcggaatccttactatcctgcaaccaactttagtacagtatatttagaagaaaatattctcatttctgatcttgagaagagtgaccaataa

SEQ ID NO: 2 - protein, 1463 aa, Homo sapiens [sequence omitted]

SEQ ID NO: 3 - DNA, 3969 bp, Homo sapiens
  CDS (1)..(3966)
atgctgctgtcgccgtcgctgctgctgctgctgctgctgggggcgccgcggggctgcgccgagggtgtggcggcggcgcttacccccgagcggctcctggagtggcaggataaaggaatatttgttatccaaagtgagagtctcaagaaatgcattcaagcaggtaaatcggttctgaccctggagaactgcaagcaagcaaacaagcacatgctgtggaaatgggtttcaaaccatggcctctttaacataggaggcagcggttgcctgggcctgaatttctccgccccagagcagccattaagcttatatgaatgtgactccaccctcgtttccttacggtggcgctgtaacaggaagatgatcacaggcccgctgcagtactctgtccaggtggcgcatgacaacacagtggtggcctcacggaagtatattcataagtggatttcttatgggtcaggtggtggagacatttgtgaatatctacacaaagatttgcatacaatcaaagggaacacccacgggatgccgtgtatgtttcccttccagtataaccatcagtggcatcatgaatgtacccgtgaaggtcgggaagatgacttactgtggtgtgccacgacaagccgttatgaaagagatgaaaagtggggattttgccctgatcccacctctgcagaagtaggttgtgatactatttgggagaaggacctcaattcacacatttgctaccagttcaacctgctttcatctctctcttggagtgaggcacattcttcatgccagatgcaaggaggtacgctgttaagtattacagatgaaactgaagaaaatttcataagggagcacatgagcagtaaaacagtggaggtgtgggtgggcctcaatcagctggatgaagacgctggctggcagtggtctgatggaacgccgctcaactatctgaattggagcccagaggtaaattttgagccatttgttgaagatcactgtggaacatttagttcatttatgccaagtgcctggaggagtcgggattgtgagtccaccttgccatatatatgtaaaaaatatctaaaccacattgatcatgaaatagttgaaaaagatgcgtggaaatattatgctacccactgtgagcctggctggaatccctacaatcgtaattgctacaaacttcagaaagaagaaaagacctggcatgaggctctgcgttcttgtcaggctgataacagtgcattaatagacataacctcattagcagaggtggagtttcttgtaaccctccttggagatgaaaatgcatcagaaacatggattggtttgagcagcaataaaattccagtttcctttgaatggtctaatgactcttcagtcatctttactaattggcacacacttgagccccacatttttccaaatagaagccagctgtgtgtctcagcagagcagtctgagggacactggaaagtcaaaaattgtgaagaaagacttttttacatttgtaaaaaagcaggccatgtcctctctgatgctgaatcaggatgtcaagagggatgggagagacatggtggattctgttacaaaattgacacagtccttcgaagctttgaccaagcttccagcggttattactgtcctcctgcacttgtaaccattacaaacaggtttgaacaggcttttattaccagtttgatcagtagtgtggtaaaaatgaaggacagttatttttggatagctcttcaggaccaaaatgatacgggagaatacacttggaagccagtagggcagaaacccgagccggtgcagtacacacactggaacacacaccagccgcgctacagtggtggctgtgttgccatgcgaggaaggcatccacttggtcgctgggaagtgaagcactgtcggcactttaaggcaatgtccttgtgcaagcagccagttgaaaatcaggaaaaagcagagtatgaagagagatggccctttcacccctgctatttggactgggagtcagagcctggtctggccagttgcttcaaggtatttcatagtgaaaaagttctgatgaaaagaacatggagagaagctgaagcattttgcgaagaatttggagctcatcttgcaagctttgcccatattgaggaagagaattttgtgaatgagctcttacattcaaaatttaattggacagaagaaaggcagttctggattggatttaataaaagaaacccactgaatgccggctcatgggagtggtctgatagaactcctgttgtctcttcgtttttagacaacacttattttggagaagatgcaagaaactgtgctgtttataaggcaaacaaaacattgctgcccttacactgtggttccaaacgtgaatggatatgcaaaatcccaagagatgtgaaacccaagattccgttctggtaccagtacgatgtaccctggctcttttatcaggatgcagaatacctttttcatacctttgcctcagaatggttgaactttgagtttgtctgtagctggctgcacagtgatcttctcacaattcattctgcacatgagcaagaattcatccacagcaaaataaaagcgctatcaaagtatggtgcaagttggtggattggacttcaagaagaaagagccaatgatgaatttcgctggagagatggaacaccagtgatataccagaactgggacacaggaagagaaagaactgtgaataatcagagccagagatgtggctttatttcttctataacaggactctggggtagtgaagagtgttcagtttctatgcctagtatctgtaagcgaaaaaaggtttggctcatagagaaaaagaaagatacaccaaaacaacatggaacgtgtcccaaaggatggctatattttaactataagtgccttctgctgaatatccccaaagacccaagcagttggaagaactggacgcatgctcaacatttctgtgctgaagaaggggggaccctggtcgccattgaaagtgaggtggagcaagctttcattactatgaatctttttggccagaccaccagtgtgtggataggtttacaaaatgatgattatgaaacatggctaaatggaaagcctgtggtatattctaactggtctccatttgatataataaatattccaagtcacaataccactgaagttcagaaacacattcctctctgtgccttactctcaagtaatcctaattttcatttcactggaaaatggtattttgaagactgtggaaaggaaggctatgggtttgtttgtgaaaaaatgcaagatacttctggacacggtgtaaatacatctgatatgtatccaatgcccaataccttagaatatggaaacagaacttacaaaataattaatgcaaatatgacttggtatgcagcaataaaaacctgcctgatgcacaaagcacaactggtcagcatcacagaccagtatcaccagtccttcctcactgttgtcctcaaccggctaggatatgcccactggattggactgttcaccacagataatggtcttaattttgactggtctgatggcaccaaatcttctttcactttttggaaagatgaggagtcctccctccttggtgactgcgtttttgccgacagcaacggacgctggcatagcacagcctgcgagtcatttctgcaaggtgccatttgtcatgtgccacctgaaacaagacaatctgaacacccagagttgtgctcagaaacatctattccctggataaaatttaaaagtaattgctacagtttttctacagtcctagacagtatgagttttgaggctgctcatgaattttgcaaaaaggaaggttctaatcttttaacaatcaaggatgaggctgaaaatgcatttctcctagaagagctgtttgcttttggttcttctgtccagatggtttggttgaatgctcaatttgatggtaactaa

SEQ ID NO: 4 - protein, 1322 aa, Homo sapiens
MLLSPSLLLLLLLGAPRGCAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWVGLNQLDEDAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGHVLSDAESGCQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQPVENQEKAEYEERWPFHPCYLDWESEPGLASCFKVFHSEKVLMKRTWREAEAFCEEFGAHLASFAHIEEENFVNELLHSKFNWTEERQFWIGFNKRNPLNAGSWEWSDRTPVVSSFLDNTYFGEDARNCAVYKANKTLLPLHCGSKREWICKIPRDVKPKIPFWYQYDVPWLFYQDAEYLFHTFASEWLNFEFVCSWLHSDLLTIHSAHEQEFIHSKIKALSKYGASWWIGLQEERANDEFRWRDGTPVIYQNWDTGRERTVNNQSQRCGFISSITGLWGSEECSVSMPSICKRKKVWLIEKKKDTPKQHGTCPKGWLYFNYKCLLLNIPKDPSSWKNWTHAQHFCAEEGGTLVAIESEVEQAFITMNLFGQTTSVWIGLQNDDYETWLNGKPVVYSNWSPFDIINIPSHNTTEVQKHIPLCALLSSNPNFHFTGKWYFEDCGKEGYGFVCEKMQDTSGHGVNTSDMYPMPNTLEYGNRTYKIINANMTWYAAIKTCLMHKAQLVSITDQYHQSFLTVVLNRLGYAHWIGLFTTDNGLNFDWSDGTKSSFTFWKDEESSLLGDCVFADSNGRWHSTACESFLQGAICHVPPETRQSEHPELCSETSIPWIKFKSNCYSFSTVLDSMSFEAAHEFCKKEGSNLLTIKDEAENAFLLEELFAFGSSVQMVWLNAQFDGN

SEQ ID NO: 5 - DNA, 3297 bp, Homo sapiens
  CDS (1)..(3294)
atgctgctgtcgccgtcgctgctgctgctgctgctgctgggggcgccgcggggctgcgccgagggtgtggcggcggcgcttacccccgagcggctcctggagtggcaggataaaggaatatttgttatccaaagtgagagtctcaagaaatgcattcaagcaggtaaatcggttctgaccctggagaactgcaagcaagcaaacaagcacatgctgtggaaatgggtttcaaaccatggcctctttaacataggaggcagcggttgcctgggcctgaatttctccgccccagagcagccattaagcttatatgaatgtgactccaccctcgtttccttacggtggcgctgtaacaggaagatgatcacaggcccgctgcagtactctgtccaggtggcgcatgacaacacagtggtggcctcacggaagtatattcataagtggatttcttatgggtcaggtggtggagacatttgtgaatatctacacaaagatttgcatacaatcaaagggaacacccacgggatgccgtgtatgtttcccttccagtataaccatcagtggcatcatgaatgtacccgtgaaggtcgggaagatgacttactgtggtgtgccacgacaagccgttatgaaagagatgaaaagtggggattttgccctgatcccacctctgcagaagtaggttgtgatactatttgggagaaggacctcaattcacacatttgctaccagttcaacctgctttcatctctctcttggagtgaggcacattcttcatgccagatgcaaggaggtacgctgttaagtattacagatgaaactgaagaaaatttcataagggagcacatgagcagtaaaacagtggaggtgtgggtgggcctcaatcagctggatgaagacgctggctggcagtggtctgatggaacgccgctcaactatctgaattggagcccagaggtaaattttgagccatttgttgaagatcactgtggaacatttagttcatttatgccaagtgcctggaggagtcgggattgtgagtccaccttgccatatatatgtaaaaaatatctaaaccacattgatcatgaaatagttgaaaaagatgcgtggaaatattatgctacccactgtgagcctggctggaatccctacaatcgtaattgctacaaacttcagaaagaagaaaagacctggcatgaggctctgcgttcttgtcaggctgataacagtgcattaatagacataacctcattagcagaggtggagtttcttgtaaccctccttggagatgaaaatgcatcagaaacatggattggtttgagcagcaataaaattccagtttcctttgaatggtctaatgactcttcagtcatctttactaattggcacacacttgagccccacatttttccaaatagaagccagctgtgtgtctcagcagagcagtctgagggacactggaaagtcaaaaattgtgaagaaagacttttttacatttgtaaaaaagcaggccatgtcctctctgatgctgaatcaggatgtcaagagggatgggagagacatggtggattctgttacaaaattgacacagtccttcgaagctttgaccaagcttccagcggttattactgtcctcctgcacttgtaaccattacaaacaggtttgaacaggcttttattaccagtttgatcagtagtgtggtaaaaatgaaggacagttatttttggatagctcttcaggaccaaaatgatacgggagaatacacttggaagccagtagggcagaaacccgagccggtgcagtacacacactggaacacacaccagccgcgctacagtggtggctgtgttgccatgcgaggaaggcatccacttggtcgctgggaagtgaagcactgtcggcactttaaggcaatgtccttgtgcaagcagccagttgaaaatcaggaaaaagcagagtatgaagagagatggccctttcacccctgctatttggactgggagtcagagcctggtctggccagttgcttcaaggtatttcatagtgaaaaagttctgatgaaaagaacatggagagaagctgaagcattttgcgaagaatttggagctcatcttgcaagctttgcccatattgaggaagagaattttgtgaatgagctcttacattcaaaatttaattggacagaagaaaggcagttctggattggatttaataaaagaaacccactgaatgccggctcatgggagtggtctgatagaactcctgttgtctcttcgtttttagacaacacttattttggagaagatgcaagaaactgtgctgtttataaggcaaacaaaacattgctgcccttacactgtggttccaaacgtgaatggatatgcaaaatcccaagagatgtgaaacccaagattccgttctggtaccagtacgatgtaccctggctcttttatcaggatgcagaatacctttttcatacctttgcctcagaatggttgaactttgagtttgtctgtagctggctgcacagtgatcttctcacaattcattctgcacatgagcaagaattcatccacagcaaaataaaagcgctatcaaagtatggtgcaagttggtggattggacttcaagaagaaagagccaatgatgaatttcgctggagagatggaacaccagtgatataccagaactgggacacaggaagagaaagaactgtgaataatcagagccagagatgtggctttatttcttctataacaggactctggggtagtgaagagtgttcagtttctatgcctagtatctgtaagcgaaaaaaggtttggctcatagagaaaaagaaagatacaccaaaacaacatggaacgtgtcccaaaggatggctatattttaactataagtgccttctgctgaatatccccaaagacccaagcagttggaagaactggacgcatgctcaacatttctgtgctgaagaaggggggaccctggtcgccattgaaagtgaggtggagcaagctttcattactatgaatctttttggccagaccaccagtgtgtggataggtttacaaaatgatgattatgaaacatggctaaatggaaagcctgtggtatattctaactggtctccatttgatataataaatattccaagtcacaataccactgaagttcagaaacacattcctctctgtgccttactctcaagtaatcctaattttcatttcactggaaaatggtattttgaagactgtggaaaggaaggctatgggtttgtttgtgaaaaataa

SEQ ID NO: 6 - protein, 1098 aa, Homo sapiens
MLLSPSLLLLLLLGAPRGCAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWVGLNQLDEDAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGHVLSDAESGCQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQPVENQEKAEYEERWPFHPCYLDWESEPGLASCFKVFHSEKVLMKRTWREAEAFCEEFGAHLASFAHIEEENFVNELLHSKFNWTEERQFWIGFNKRNPLNAGSWEWSDRTPVVSSFLDNTYFGEDARNCAVYKANKTLLPLHCGSKREWICKIPRDVKPKIPFWYQYDVPWLFYQDAEYLFHTFASEWLNFEFVCSWLHSDLLTIHSAHEQEFIHSKIKALSKYGASWWIGLQEERANDEFRWRDGTPVIYQNWDTGRERTVNNQSQRCGFISSITGLWGSEECSVSMPSICKRKKVWLIEKKKDTPKQHGTCPKGWLYFNYKCLLLNIPKDPSSWKNWTHAQHFCAEEGGTLVAIESEVEQAFITMNLFGQTTSVWIGLQNDDYETWLNGKPVVYSNWSPFDIINIPSHNTTEVQKHIPLCALLSSNPNFHFTGKWYFEDCGKEGYGFVCEK

SEQ ID NO: 7 - DNA, 1938 bp, Homo sapiens
  CDS (1)..(1935)
atgctgctgtcgccgtcgctgctgctgctgctgctgctgggggcgccgcggggctgcgccgagggtgtggcggcggcgcttacccccgagcggctcctggagtggcaggataaaggaatatttgttatccaaagtgagagtctcaagaaatgcattcaagcaggtaaatcggttctgaccctggagaactgcaagcaagcaaacaagcacatgctgtggaaatgggtttcaaaccatggcctctttaacataggaggcagcggttgcctgggcctgaatttctccgccccagagcagccattaagcttatatgaatgtgactccaccctcgtttccttacggtggcgctgtaacaggaagatgatcacaggcccgctgcagtactctgtccaggtggcgcatgacaacacagtggtggcctcacggaagtatattcataagtggatttcttatgggtcaggtggtggagacatttgtgaatatctacacaaagatttgcatacaatcaaagggaacacccacgggatgccgtgtatgtttcccttccagtataaccatcagtggcatcatgaatgtacccgtgaaggtcgggaagatgacttactgtggtgtgccacgacaagccgttatgaaagagatgaaaagtggggattttgccctgatcccacctctgcagaagtaggttgtgatactatttgggagaaggacctcaattcacacatttgctaccagttcaacctgctttcatctctctcttggagtgaggcacattcttcatgccagatgcaaggaggtacgctgttaagtattacagatgaaactgaagaaaatttcataagggagcacatgagcagtaaaacagtggaggtgtgggtgggcctcaatcagctggatgaagacgctggctggcagtggtctgatggaacgccgctcaactatctgaattggagcccagaggtaaattttgagccatttgttgaagatcactgtggaacatttagttcatttatgccaagtgcctggaggagtcgggattgtgagtccaccttgccatatatatgtaaaaaatatctaaaccacattgatcatgaaatagttgaaaaagatgcgtggaaatattatgctacccactgtgagcctggctggaatccctacaatcgtaattgctacaaacttcagaaagaagaaaagacctggcatgaggctctgcgttcttgtcaggctgataacagtgcattaatagacataacctcattagcagaggtggagtttcttgtaaccctccttggagatgaaaatgcatcagaaacatggattggtttgagcagcaataaaattccagtttcctttgaatggtctaatgactcttcagtcatctttactaattggcacacacttgagccccacatttttccaaatagaagccagctgtgtgtctcagcagagcagtctgagggacactggaaagtcaaaaattgtgaagaaagacttttttacatttgtaaaaaagcaggccatgtcctctctgatgctgaatcaggatgtcaagagggatgggagagacatggtggattctgttacaaaattgacacagtccttcgaagctttgaccaagcttccagcggttattactgtcctcctgcacttgtaaccattacaaacaggtttgaacaggcttttattaccagtttgatcagtagtgtggtaaaaatgaaggacagttatttttggatagctcttcaggaccaaaatgatacgggagaatacacttggaagccagtagggcagaaacccgagccggtgcagtacacacactggaacacacaccagccgcgctacagtggtggctgtgttgccatgcgaggaaggcatccacttggtcgctgggaagtgaagcactgtcggcactttaaggcaatgtccttgtgcaagcagtaa

SEQ ID NO: 8 - protein, 645 aa, Homo sapiens
MLLSPSLLLLLLLGAPRGCAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDPTSAEVGCDTIWEKDLNSHICYQFNLLSSLSWSEAHSSCQMQGGTLLSITDETEENFIREHMSSKTVEVWVGLNQLDEDAGWQWSDGTPLNYLNWSPEVNFEPFVEDHCGTFSSFMPSAWRSRDCESTLPYICKKYLNHIDHEIVEKDAWKYYATHCEPGWNPYNRNCYKLQKEEKTWHEALRSCQADNSALIDITSLAEVEFLVTLLGDENASETWIGLSSNKIPVSFEWSNDSSVIFTNWHTLEPHIFPNRSQLCVSAEQSEGHWKVKNCEERLFYICKKAGHVLSDAESGCQEGWERHGGFCYKIDTVLRSFDQASSGYYCPPALVTITNRFEQAFITSLISSVVKMKDSYFWIALQDQNDTGEYTWKPVGQKPEPVQYTHWNTHQPRYSGGCVAMRGRHPLGRWEVKHCRHFKAMSLCKQ

SEQ ID NO: 9 - DNA, 669 bp, Homo sapiens
  CDS (1)..(666)
atgctgctgtcgccgtcgctgctgctgctgctgctgctgggggcgccgcggggctgcgccgagggtgtggcggcggcgcttacccccgagcggctcctggagtggcaggataaaggaatatttgttatccaaagtgagagtctcaagaaatgcattcaagcaggtaaatcggttctgaccctggagaactgcaagcaagcaaacaagcacatgctgtggaaatgggtttcaaaccatggcctctttaacataggaggcagcggttgcctgggcctgaatttctccgccccagagcagccattaagcttatatgaatgtgactccaccctcgtttccttacggtggcgctgtaacaggaagatgatcacaggcccgctgcagtactctgtccaggtggcgcatgacaacacagtggtggcctcacggaagtatattcataagtggatttcttatgggtcaggtggtggagacatttgtgaatatctacacaaagatttgcatacaatcaaagggaacacccacgggatgccgtgtatgtttcccttccagtataaccatcagtggcatcatgaatgtacccgtgaaggtcgggaagatgacttactgtggtgtgccacgacaagccgttatgaaagagatgaaaagtggggattttgccctgatccctaa

SEQ ID NO: 10 - protein, 222 aa, Homo sapiens
MLLSPSLLLLLLLGAPRGCAEGVAAALTPERLLEWQDKGIFVIQSESLKKCIQAGKSVLTLENCKQANKHMLWKWVSNHGLFNIGGSGCLGLNFSAPEQPLSLYECDSTLVSLRWRCNRKMITGPLQYSVQVAHDNTVVASRKYIHKWISYGSGGGDICEYLHKDLHTIKGNTHGMPCMFPFQYNHQWHHECTREGREDDLLWCATTSRYERDEKWGFCPDP

SEQ ID NO: 11 - DNA, 34 bp, Artificial Sequence
  Forward primer
tactcgagccaccatgctgctgtcgccgtcgctg

SEQ ID NO: 12 - DNA, 34 bp, Artificial Sequence
  WT reverse primer
tagcggccgcttattggtcactcttctcaagatc

SEQ ID NO: 13 - DNA, 33 bp, Artificial Sequence
  FNII reverse primer
tagcggccgcttagggatcagggcaaaatcccc

SEQ ID NO: 14 - DNA, 34 bp, Artificial Sequence
  CTLD3 reverse primer
tagcggccgcttactgcttgcacaaggacattgc

SEQ ID NO: 15 - DNA, 40 bp, Artificial Sequence
  CTLD6 reverse primer
tagcggccgcttatttttcacaaacaaacccatagccttc

SEQ ID NO: 16 - DNA, 43 bp, Artificial Sequence
  CTLD8 reverse primer
tagcggccgctcacttactgttaccatcaaattgagcattcaa